(19) United States
(12) Patent Application Publication
(10) Pub. No.: US 2023/0340395 A1
(43) Pub. Date: Oct. 26, 2023

(54) SYSTEM FOR TREATING A BIOTECHNOLOGICAL FLUID

(71) Applicant: MERCK PATENT GMBH, DARMSTADT (DE)

(72) Inventors: Rene REINBIGLER, Molsheim (FR); Denis DUBRET, Molsheim (FR); Steven MILLER, Molsheim (FR)

(73) Assignee: MERCK PATENT GMBH, DARMSTADT (DE)

(21) Appl. No.: 18/022,347

(22) PCT Filed: Aug. 19, 2021
(86) PCT No.: PCT/EP2021/072988

§ 371 Date: Feb. 21, 2023

(30) Foreign Application Priority Data
Aug. 22, 2020 (EP) 20305947.2

(51) Int. Cl. C12M 1/34; C12M 1/36

(52) U.S. Cl. CPC C12M 41/48; C12M 41/26; C12M 41/42

(57) ABSTRACT

The present application relates to biotechnological fluid treating, such as biopharmaceutical liquids in order to obtain products such as monoclonal antibodies, vaccines or recombinant proteins, and particularly to a system for treating a biotechnical fluid, having a bioprocess machine and at least one bioprocess machine helper, configured to connect to each other and each comprising a controller, in turn comprising a machine-to-machine communication tool configured for connecting to a network

### FIELD OF THE INVENTION

The invention relates to biotechnological fluid treating, such as biopharmaceutical liquids in order to obtain products such as monoclonal antibodies, vaccines or recombinant proteins.

### BACKGROUND ART

It is known that biotechnological fluids such as biopharmaceutical liquids are in general obtained first by a treatment such as cell or micro-organism culture in a bioreactor and that they must then be further treated to achieve the required characteristics of homogeneity, purity, concentration, absence of viruses, etc.

These treatments are conventionally carried out in dedicated installations, with stainless steel pipes and other components such as tanks and filter housings, which necessitate operations before and after the actual treatment, which are relatively onerous, in particular, operations for cleaning after use.

Within the last few years, these treatments have alternatively been carried out in installations in which the components in contact with the liquid are single-use components, for avoiding cleaning operations.

For instance, European patent applications EP 2 130 903 and EP 2 208 534 disclose installations including disposable elements, for the most part flexible (“Flexware™ products”), including the treated liquid collecting bag and the circuit sections, even the filter element, and permanent or reusable elements (“hardware”) accommodated in two or more carts, so that an installation for treating a biotechnological fluid can be assembled simply by equipping the carts with the disposable elements, whereas the post-treatment steps are essentially the removal and discarding of the disposable elements.

Other known installations are using the same approach with the reusable elements or certain reusable elements which are not accommodated in carts.

In general, the main reusable element of an installation for treating a biotechnological fluid is a bioprocess machine having a biotechnological fluid treater configured for modifying at least one physico-chemical or biological property of the biotechnological fluid, for example its pH, DO (Dissolved Oxygen), homogeneity, purity, concentration, presence or absence of predetermined micro-organisms, e.g., viruses and/or other pathogens.

Further to the biotechnological fluid treater, the bioprocess machine has a digital controller for controlling the biotechnological fluid treater and most often the digital controller is able to pilot the fluid treater so that the machine can carry out automatically a customized version of the treatment, generally called a recipe.

### SUMMARY OF THE INVENTION

The invention is directed to further ease the setting up of installations for treating a biotechnological fluid.

The invention provides accordingly a system having the following system devices: a bioprocess machine (13) having: a biotechnological fluid treater (15) configured for modifying at least one physico-chemical or biological property of said biotechnological fluid and a digital controller (16) for controlling said biotechnological fluid treater (15); and at least one bioprocess machine helper (14) having: a biotechnological fluid treater helper (21) configured for being physically coupled to said biotechnological fluid treater (15) and a digital controller (22) for controlling said biotechnological fluid treater helper (21); wherein said digital controller (16) of said bioprocess machine (13) and said digital controller (22) of said machine helper (14) each include a recipe manager (50, 51), a machine to machine communication tool (18, 24) (MtoM), and a discovery negotiation pairing manager (19, 25) (DNP manager); each said MtoM communication tool (18, 24) is configured for connecting to a network (12); the DNP manager (19) of said bioprocess machine (13) and the DNP manager (25) of said machine helper (14) are configured for cooperating over said network (12) for establishing a paired condition; wherein the recipe manager (50) of the bioprocess machine (13) and the recipe manager (51) of the machine helper (14) are configured such that in said paired condition: the recipe manager (50) of the bioprocess machine (13) has at least one provided capability that it does not have when not in paired condition, said provided capability being an automation function controlling and/or testing an operating parameter of said treater helper (21) or testing a physico-chemical or biological quantity of said fluid sensed by said treater helper (21); and the recipe manager (51) of the machine helper (14) has at least one consumed capability that it does not have when not in paired condition, wherein: if said provided capability is said automation function controlling and/or testing an operating parameter of the treater helper (21) said consumed capability is a response-on-request function controlling and/or making available said operating parameter, and if said provided capability is said automation function testing a physico-chemical or biological quantity of said fluid sensed by the treater helper (21) said consumed capability is a response-on-request function making available said physico-chemical or biological quantity.

The physical coupling between the machine helper (through the treater helper) and the bioprocess machine (through the fluid treater) is of course for enabling the treater helper to assist the fluid treater in modifying the at least one physico-chemical or biological property of the biotechnological fluid, for example by pumping or exerting another physical action on the fluid or by sensing a physico-chemical or biological quantity of the fluid, such as pH or Dissolved Oxygen (DO).

In the system according to the invention, the machine helper has a digital controller with a machine to machine communication tool so as to communicate with the bioprocess machine through a network enabling machine to machine communications, despite the physical coupling between the machine helper and the bioprocess machine which would have enabled relatively easily a dedicated communication channel such as a wired serial or parallel link, which could be operational merely by plugging connectors when carrying out the physical coupling.

The invention is based on the observation that despite the need to carry out pairing through the network further to the physical coupling, such pairing through the network can in fact ease the setting up of installations for treating a biotechnological fluid because such pairing can be used for automatically reconfiguring accordingly the bioprocess machine (with the provided capability) and the machine helper (with the consumed capability).

With such automated reconfiguring, adding a machine helper is very convenient and can be done even during a batch process so that the system according to the invention further offers excellent flexibility.

According to advantageous features for carrying out the system according to the invention, the recipe manager (50) of the bioprocess machine (13) is configured for displaying a graphical user interface, said graphical user interface of the recipe manager (50) of the bioprocess machine (13) being configured for allowing a user to create recipes with said provided capability automation function, to store so created recipes, to load so stored recipes and to execute so loaded recipes;
wherein said recipe manager (51) of the machine helper (14) is not configured for displaying a graphical user interface;
wherein said provided capability automation function is controlling and/or testing a set of operating parameters of said treater helper (21) or testing a set of physico-chemical or biological quantity of said fluid sensed by said treater helper (21);
wherein said digital controller (16) of said bioprocess machine (13) includes a file (20) containing a description of each said automation function which can be a said provided capability and said digital controller (22) of said machine helper (14) includes a file (26) containing a description of each said response-on-request function which can be a said consumed capability;
wherein the DNP manager (19) of said bioprocess machine (13) and the DNP manager (25) of said machine helper (14) are configured for cooperating over said network (12) for establishing a paired condition;
wherein said system devices include a plurality of said machine helpers (14) and the DNP manager (19) of said bioprocess machine (13) is configured for establishing a said paired condition simultaneously with at least two said machine helpers (14);
wherein said system devices include a first said machine helper (14) and a second said machine helper (14); the fluid treater helper (21) of the first machine helper (14) and the fluid treater helper (21) of the second machine helper (14) are configured for being physically coupled one another; and the DNP manager (25) of said first machine helper (14) and the DNP manager (25) of said second machine helper (14) are configured for cooperating over said network (12) for establishing a paired condition; wherein the recipe manager (51) of the first machine helper (14) and the recipe manager (51) of the second machine helper (14) are configured such that in said paired condition: the recipe manager (51) of the first machine helper (14) has at least one provided capability that it does not have when not in paired condition, said provided capability being a response-on-request function controlling and/or testing an operating parameter of the treater helper (21) of the second machine helper (14) or testing a physico-chemical or biological quantity of said fluid sensed by the treater helper (21) of the second machine helper (14); and the recipe manager (51) of the second machine helper (14) has at least one consumed capability that it does not have when not in paired condition, wherein: if said provided capability is said response-on-request function controlling and/or testing an operating parameter of the treater helper (21) of the second machine helper (14) said consumed capability is a response-on-request function controlling and/or making available said operating parameter, and if said provided capability is said response-on-request function testing a physico-chemical or biological quantity of said fluid sensed by the treater helper (21) of the second machine helper (14) said consumed capability is a response-on-request function making available said physico-chemical or biological quantity; the recipe manager wherein the recipe manager (50) of said bioprocess machine (13), the recipe manager (51) of said first machine helper (14) and the recipe manager (51) of said second machine helper (14) are configured such that in said paired condition of the bioprocess machine (13) with the first machine helper (14) while the first machine helper (14) is in paired condition with the second machine helper (14) and while the bioprocess machine (13) is in paired condition with the second machine helper (14), the recipe manager (50) of the bioprocess machine (13) has the same provided capability as the provided capability in the first machine helper (14), said same provided capability and said provided capability in the first machine helper (14) corresponding to said consumed capability in the second machine helper (14);
wherein the recipe manager (50) of said bioprocess machine (13) and the recipe manager (51) of said first machine helper (14) are configured such that in said paired condition of the bioprocess machine with the first machine helper (14) while the first machine helper (14) is in paired condition with the second machine helper (14), the reporting manager (60) of the bioprocess machine (13) has a provided capability replicating the provided capability in the first machine helper (14) corresponding to the consumed capability in the second machine helper (14);
wherein the reporting manager (60) of said bioprocess machine (13) and the recipe manager (51) of said first machine helper (14) are configured such that in said paired condition of the bioprocess machine with the first machine helper (14) while the first machine helper (14) is in paired condition with the second machine helper (14), the reporting manager (60) of the bioprocess machine (13) has a provided capability embedding the provided capability in the first machine helper (14) corresponding to the consumed capability in the second machine helper (14);
wherein said system devices include a first said bioprocess machine (13), a second said bioprocess machine (13) and a plurality of said machine helpers (14); and the DNP manager (25) of at least one said machine helper (14) is configured for establishing a said paired condition either with said first bioprocess machine (13) or with said second bioprocess machine (13); having a first said machine helper (14) in which the consumed capability is a response-on-request function controlling and/or making available an operating parameter of the treater helper (21) of said first machine helper (14); and having a second said machine helper (14) in which the consumed capability is a response-on-request function making available a physico-chemical or biological quantity of the fluid sensed by the treater helper (21) of second machine helper (14), wherein the first machine helper (14) is a pump in which the consumed capability is a request-on-response function controlling and/or making available a speed of the pump and the second machine helper (14) is a pH sensor or a flow sensor in which the consumed capability is a response-on-request function making available a pH of said fluid or a flow of said fluid;
wherein each said MtoM communication tool (18, 24) is configured for connecting to a said network (12) which is a network with Internet Protocol, such as Ethernet, Wi-Fi, Bluetooth or cellular 5G; and/or in said system, wherein:
said digital controller (16) of said bioprocess machine (13) and said digital controller (22) of said machine helper (14) each further include a graphical user interface manager (17, 23) (GUI manager)
the GUI manager (17) of the bioprocess machine (13) and the GUI manager (23) of the machine helper (14) are configured such that in said paired condition: the GUI manager (17) of the bioprocess machine (13) has at least one provided capability that it does not have when not in paired condition, said provided capability being an interface function controlling and/or displaying an operating parameter of said treater helper (21) or displaying a physico-chemical or biological quantity of said fluid sensed by said treater helper (21); and the GUI manager (23) of the machine helper (14) has at least one consumed capability which is modified with respect to when not in paired condition, wherein: if said provided capability is said interface function controlling and/or displaying an operating parameter of the treater helper (21) said consumed capability is an interface function controlling and/or displaying said operating parameter, and if said provided capability is said interface function displaying a physico-chemical or biological quantity of said fluid sensed by the treater helper (21) said consumed capability is an interface function displaying said physico-chemical or biological quantity.

### BRIEF DESCRIPTION OF THE DRAWINGS

The description of the invention continues now with a detailed description of example embodiments given hereinafter by way of non-limiting illustration and with reference to the appended drawings. In the drawings:

FIG. 1 schematically illustrates a production area in a bioprocess production plant or laboratory, in which is located a bioprocess machine forming part of a system for treating a biological fluid;

FIG. 2 schematically illustrates a storage area of the bioprocess production plant or laboratory, in which are located a plurality of bioprocess machine helpers of the system for treating a biological fluid, the bioprocess machine helpers being configured for being associated to the bioprocess machine illustrated on FIG. 1;

FIG. 3 is a diagram illustrating the bioprocess machine and one of the bioprocess machine helpers, together with a network through which the bioprocess machine and the bioprocess machine helper cooperate for establishing a paired condition;

FIG. 4 shows the graphical user interface of a bioprocess machine which is a mixer, in stand-alone condition;

FIG. 5 shows at the top the graphical user interface of the mixer when paired with a bioprocess machine helper which is a pH sensor, and shows at the bottom the graphical user interface of the pH sensor, respectively on the left in stand-alone condition and on the right when paired with the mixer;

FIG. 6 schematically illustrates at the top a bioprocess machine helper which is a pump together with a bioprocess machine helper which is a flow sensor, the pump and the flow sensor being physically coupled; and also shows at the middle the graphical user interface of the pump, respectively on the left in stand-alone condition and on the right when paired with the flow sensor; and also shows at the bottom the graphical user interface of the flow sensor, respectively on the left in stand-alone condition and on the right when paired with the pump;

FIG. 7 is an exemplary schematic representation of the GUIs adapting when the process machine and a machine helper are being paired, showing at the top the graphical user interface of the mixer, respectively on the left when paired with the pH sensor and on the right when paired with the pH sensor and paired with the pump paired with the flow sensor; and also showing at the middle the graphical user interface of the pump, respectively on the left when paired with the flow sensor and on the right when paired with the flow sensor and paired with the mixer; and also showing at the bottom the graphical user interface of the flow sensor, respectively on the left when paired with the pump, and on the right when paired with the pump paired with the mixer;

FIG. 8 is an exemplary schematic representation of the GUIs adapting when the process machine and a machine helper are being unpaired, showing at the top the graphical user interface of the mixer, respectively on the left when paired with the pH sensor and paired with the pump paired with the flow sensor, and on the right when paired with the pH sensor; and also showing at the middle the graphical user interface of the pump, respectively on the left when paired with the flow sensor and paired with the mixer and on the right when paired with the flow sensor; and also showing at the bottom the graphical user interface of the flow sensor, respectively on the left when paired with the pump paired with the mixer and on the right when paired with the pump;

FIG. 9 is a diagram similar to FIG. 3 but for a variant of the system in which the digital controller of the bioprocess machine and the digital controller of the machine helper each further includes a recipe manager;

FIG. 10 is a diagram showing certain modules of the recipe manager of the bioprocess machine;

FIG. 11 shows the graphical user interface of the recipe manager of a bioprocess machine which is the mixer, in stand-alone condition;

FIG. 12 is similar to FIG. 11, but in paired condition of the mixer with a machine helper which is the pH sensor; and

FIG. 13 is similar to FIGS. 11 and 12, but in paired condition of the mixer with the pH sensor and with the pump paired with the flow sensor.

### DETAILED DESCRIPTION OF EXAMPLE EMBODIMENTS

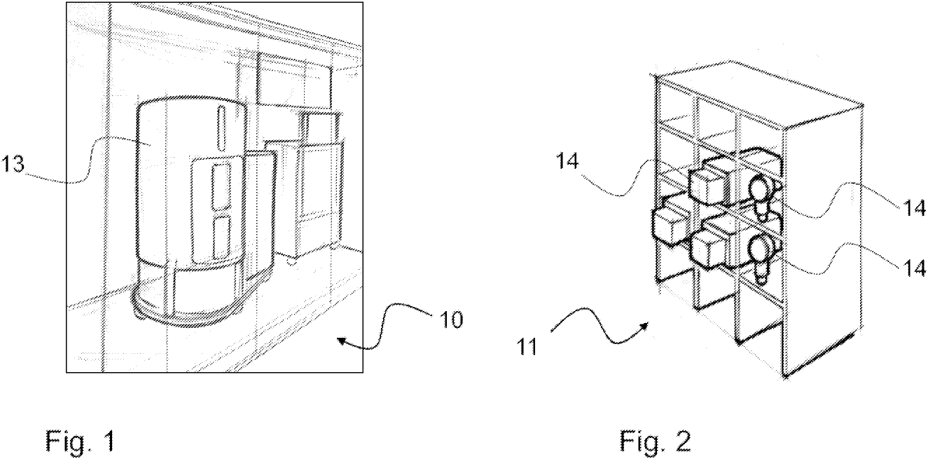

FIGS. 1 and 2 illustrate the production area 10 and the storage area 11 of a bioprocess production plant or laboratory in which are available system devices of a system for treating a biotechnological fluid according to the invention.

### Description of the System Devices: Bioprocess Machine and Bioprocess Machine Helper

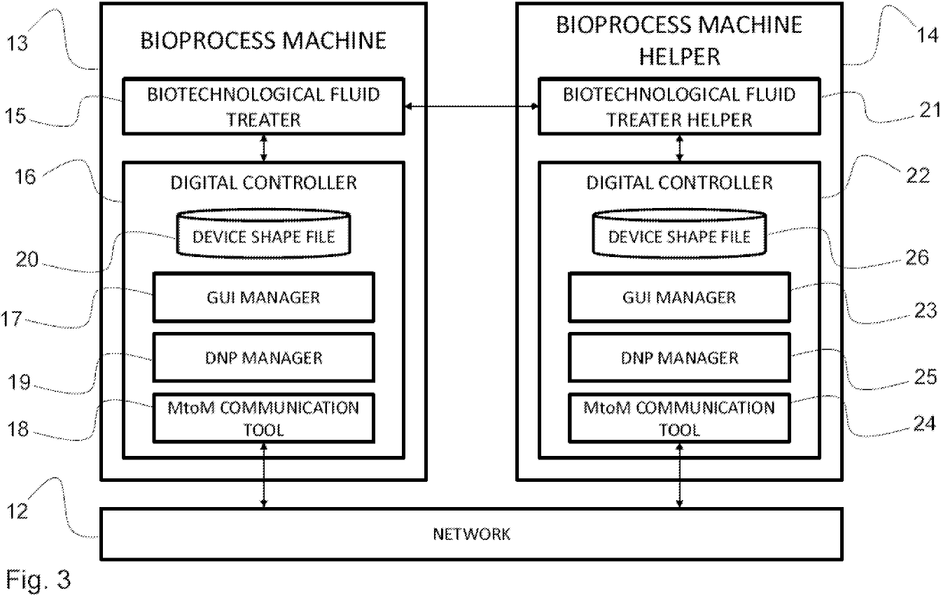

Each system device includes digital processing units (microprocessor and/or microcontroller, memory, network connectivity) and is configured to be wire or wireless connected to a network 12 (FIG. 3) supporting Internet Protocol (IP).

For instance, wire connection is through Ethernet and wireless connection is through Wi-Fi, Bluetooth or cellular such as 5G.

This system has a bioprocess machine 13 and a plurality of bioprocess machine helpers 14. The bioprocess machine 13 alone or associated with one or more machine helpers 14 can be set up for becoming an installation for treating a biotechnological fluid.

As shown on FIG. 3, the bioprocess machine 13 has a biotechnological fluid treater 15 and a digital controller 16.

The biotechnological fluid treater 15 is configured for modifying at least one physico-chemical or biological property of the biotechnological fluid, for example its pH, DO (Dissolved Oxygen), homogeneity, purity, concentration, presence or absence of predetermined micro-organisms such as viruses.

The digital controller 16 is configured for controlling the biological fluid treater 15, as shown by a bidirectional arrow on FIG. 3. Here, the digital controller 16 is able to pilot the fluid treater 15 so that the machine 13 can carry out automatically a customized version of the treatment, generally called a recipe.

The digital controller 16 includes a graphical user interface (GUI) manager 17, a machine to machine (MtoM) communication tool 18, and a discovery negotiation pairing (DNP) manager 19. The digital controller 16 also includes a file 20 called Device Shape which contains a description of certain interface functions of a GUI which can be displayed by the GUI manager 17.

It should be noted that the term “file” is here to be taken in a broad sense, namely encompassing any structured data container, including a folder and/or a database.

The bioprocess machine helper 14 has a biotechnological fluid treater helper 21 and a digital controller 22.

The treater helper 21 is configured for being physically coupled to the fluid treater 15, as shown by a bidirectional arrow on FIG. 3. The physical coupling is for enabling the treater helper 21 to assist the fluid treater 15 in modifying at least one physico-chemical or biological property of the biotechnological fluid, for example by pumping or exerting another physical action on the fluid or by sensing a physico-chemical or biological quantity of the fluid, such as pH or DO.

For instance, if the bioprocess machine 13 is a mixer, the biotechnological fluid treater 15 includes the tank and the agitator; if the machine helper 14 is a pump, the biotechnological fluid treater helper 21 includes the fluid driving member(s) such as the roller(s) of a peristaltic pump; and if the machine helper 14 is pH or flow sensor, the biotechnological fluid treater helper 21 includes respectively a pH probe and a flow probe.

The physical coupling between the fluid driving member(s) (treater helper 21 of the pump) and the tank+agitator (fluid treater 15 of the mixer) involves pipes and also holders for maintaining in a predetermined relative position the fluid driving member(s) and the tank+agitator. For instance, such holders are carried out through mounting of the pump on the same or similar framework as the mixer or through a cart, on which is mounted the pump, such cart being maintained in a fixed position with respect to the mixer.

Similarly, the pH probe or flow probe must interact with the fluid and be maintained in place.

Generally speaking, the physical coupling involves an interaction with the fluid (with or without contact, see the rollers of a peristaltic pump which are not in contact with the fluid or an IR probe of a temperature sensor which is not in contact with the fluid); and holders for maintaining the treater helper 21 with respect to the fluid treater 15.

The digital controller 22 is configured for controlling the treater helper 21, as shown by a bidirectional arrow on FIG. 3.

The digital controller 22 has the same architecture as the digital controller 16: the digital controller 22 includes a GUI manager 23, a MtoM communication tool 24, and a DNP manager 25. The digital controller 22 also includes a file 26 called Device Shape which contains a description of certain interface functions of a GUI which can be displayed by the GUI manager 23.

In each system device 13 or 14, the GUI manager 17 or 23 allows to display a GUI such as a Process and Instrumentation Diagram (P&ID) locally on an interactive screen or remotely on a device having an interactive screen, for e.g., a tablet or smartphone.

In each system device 13 or 14, the MtoM communication tool 18 or 24 is configured for connecting to the network 12, as shown by bidirectional arrows on FIG. 3.

The DNP manager 19 of the bioprocess machine 13 and the DNP manager 25 of the machine helper 14 are configured for cooperating over the network 12 for establishing a paired condition.

Each system device 13 or 14 can be used as a stand-alone device or paired with an appropriate other system device. The GUI manager 17 or 23 of each system device 13 or 14 is configured for undergoing an adaptation of the graphical user interface (GUI) when the system device transitions from a stand-alone (not paired) condition to a paired condition and vice-versa.

For instance, if the machine helper 14 is a pump which can be paired with a bioprocess machine 13, in stand-alone condition of the pump its GUI enables the user to control the pump so that it is possible for the user to utilize the pump as a stand-alone unit for tasks such as transferring a liquid from one tank to another; whereas in paired condition of the pump with the bioprocess machine 13 the GUI of the pump no longer enables the user to control the pump, only the GUI of the bioprocess machine 13 enables to control the pump.

Still for instance, if the machine helper 14 is a sensor which can be paired with a bioprocess machine 13, such sensor sensing a physico-chemical or biological quantity of a biotechnological fluid, in stand-alone condition of the sensor its GUI displays the sensed quantity so that it is possible for the user to utilize the sensor as a stand-alone unit, whereas in paired condition of the sensor with the bioprocess machine 13 the GUI of the sensor displays only a message, such as “CONNECTED”, informing that the sensor is in paired condition and the GUI of the bioprocess machine 13 displays the quantity sensed by the sensor.

Generally speaking, the GUI manager 17 of the bioprocess machine 13 and the GUI manager 23 of the machine helper 14 are configured such that in paired condition:
the GUI manager 17 of the bioprocess machine 13 has at least one provided capability that it does not have when not in paired condition, said provided capability being an interface function controlling and/or displaying an operating parameter of the treater helper 21 or displaying a physico-chemical or biological quantity of the fluid sensed by the treater helper 21; and
the GUI manager 23 of the machine helper 14 has at least one consumed capability which is modified with respect to when not in paired condition, wherein: if said provided capability is said interface function controlling and/or displaying an operating parameter of the treater helper 21 said consumed capability is an interface function controlling and/or displaying said operating parameter, and if said provided capability is said interface function displaying a physico-chemical or biological quantity of said fluid sensed by the treater helper 21 said consumed capability is an interface function displaying said physico-chemical or biological quantity.

### Capabilities

The interface functions described in the Device Shape file 20 or 26 of the different system devices 13 and 14 are either of a first type or of a second type.

The interface functions of the first type are interface functions that the GUI manager 17 or 23 of the system device 13 or 14 does not have when the system device is not paired with an appropriate other system device but with which the GUI manager 17 or 23 is supplemented when the system device 17 or 23 is paired with the appropriate other system device.

In practice, interface functions of the first type are present but disabled when the system device 13 or 14 is not paired with an appropriate other system device and enabled when the system device 13 or 14 is paired with an appropriate other system device.

When enabled, each such interface function controls and/or displays an operating parameter of a paired system device or displays a quantity sensed by a paired system device, said quantity being a physico-chemical or biological quantity of the biotechnological fluid being treated.

For mere language convenience, such interface functions are designated herein as a “capability” and when enabled as a “provided capability.”

The interface functions of the second type are interface functions that the GUI manager 23 of a system device which is a machine helper 14 has in an original form when the system device is not paired with an appropriate other system device and in a modified form when the system device is paired with the appropriate other system device.

When in original form, each such interface function controls and/or displays an operating parameter of the system device or displays a quantity sensed by a paired system device, said quantity being a physico-chemical or biological quantity of the biotechnological fluid being treated. When in modified form, each such interface function is for instance as in original form but with an additional display of an indication that the system device is paired with an appropriate other system device, or the original form is replaced by an indication that the system device is paired with an appropriate other system device, such indication being for instance an icon, a message or the absence of display.

For mere language convenience, such interface functions are designated herein as a “capability” and when in modified form as a “consumed capability.”

It should be noted that the Device Shape file 20 of the bioprocess machine 13 contains a description of interface functions of its GUI manager 17 which are all of the first type; and that the Device Shape file 26 of a machine helper 14 contains a description of at least one interface function of its GUI manager 23 which is of the second type.

It should be further noted that in the Device Shape files 20 and 26, each capability description has a feature named “Role” identifying whether the capability is of the first type or of the second type.

For the first type, the Role feature is at “Consumer”, with reference to the corresponding capability in the paired system device which becomes a “consumed capability” in paired condition. A system device 13 or 14 having a capability with the Role feature at “Consumer” is mentioned herein as a being a capability consumer.

For the second type, the Role feature is at “Provider”, with reference to the corresponding capability in the paired system device which becomes a “provided capability” in paired condition. A system device 14 having a capability with the Role feature at “Provider” is mentioned herein as a being a capability provider.

It should be noted that there is always a correspondence between a provided capability and a consumed capability.

When the provided capability (in the capability consumer 13 or 14) is an interface function controlling and/or displaying an operating parameter of the capability provider, the consumed capability (in the capability provider 14) is an interface function controlling and/or displaying this operating parameter. For instance, if the provided capability in a mixer is a Start/Stop control of a paired pump, the consumed capability in the paired pump is a Start/Stop control of this pump.

When the provided capability (in the capability consumer 13 or 14) is an interface function displaying a physico-chemical or biological quantity of the biotechnological fluid sensed by the capability provider 14, the consumed capability (in the capability provider 14) is an interface function displaying this physico-chemical or biological quantity. For instance, if the provided capability in a mixer is a display of the pH of the biotechnological fluid sensed by a paired pH sensor, the consumed capability in the paired pH sensor is the display of the sensed pH.

Corresponding provided capability and consumed capability are referred to herein as “shared capabilities.”

For each system device 13 or 14, the Device Shape file 20 or 26 is deployed at design time and can be updated, at runtime, and during the life of the system device, allowing to extend the list of capabilities a capability consumer can consume or a capability provider can provide. This renders it possible to make the system devices contribute to a new platform feature, without modifying (and then requalifying) the software package installed in the system devices.

Now will be described examples of system devices 13 and 14 and how an installation for treating a biotechnological fluid is set up with these system devices.

The example of bioprocess machine 13 is a mixer (capability consumer). The examples of bioprocess machine helpers 14 are a pH sensor (capability provider), a flow sensor (capability provider) and a pump (together capability consumer and capability provider).

### Description of the Mixer

The mixer includes a tank, an agitator within the tank and two inlets allowing to connect pipes that can be used to fill the tank.

The tank, agitator and inlets form a biotechnological fluid treater 15.

To be operational, the mixer requires connection to at least one pump to flow the biotechnological fluid through one of the inlets.

The mixer includes digital processing units including an industrial Programmable Logic Controller (PLC) and an industrial PC.

The PLC is dedicated to the real time control and monitoring of the different equipment modules to which it is connected (for example, in a wireless manner or by wire), such as the agitator and valves opening or closing the inlets.

On the industrial PC is installed a software package and stored the file 20 called Device Shape.

The digital processing units, the installed software package and the stored Device Shape file 20 form a digital controller 16.

The installed software package includes: a DNP manager 19; a MtoM communication tool 18 having here an OPC UA server and an OPC UA client to support data exchanges with other system devices; and a GUI manager 17 that allows to display a process and instrumentation diagram (P&ID) locally on an interactive screen or remotely on a device having an interactive screen, for e.g., a tablet or smartphone.

The file 20 called Device Shape contains a description of four interface functions which are provided capabilities when enabled.

### Description of the Capabilities Which Can Be Utilized by the Software Package Of the Mixer

As just mentioned, there are four such capabilities, respectively Interface Function 1, Interface Function 2, Interface Function 3 and Interface Function 4.

### Interface Function 1

When enabled, Interface Function 1 supplements the P&ID GUI with process data provided by paired system devices, whatever the paired system devices are.

The capability Interface Function 1 has the following description in the Device Shape file 20 of the mixer: Domain: “Graphics”, Purpose: “Process Value Display”, Role: “Consumer”, Restriction/Condition: optional. List of properties:

| | |
|---|---|
| Property | Attributes |
| process_value | mandatory=yes, type=decimal, access=OpcUa |
| process_value_name | mandatory=yes, type=text, access=OpcUa |
| process_value_unit | mandatory=yes, type=text, access=OpcUa |
| significant_decimal_digits | mandatory=no, type=decimal, access=OpcUa |

This capability description means: the mixer, as a capability consumer with graphic skills, is able to display process value from several paired system devices if these paired system devices provide at least the process value, the process value name and unit and optionally the number of significant decimal digits using the OPC UA standard. No restriction or condition are imposed for the negotiation or pairing.

In a variant, the list of properties further includes at least one of:

| | |
|---|---|
| Property | Attributes |
| process_value_min_range | mandatory=no, type=no, access=OpcUa |
| process_value_max_range | mandatory=no, type=no, access=OpcUa |

In such a variant, the capability description further means that the mixer is able to use the process value range if provided by a paired system device, to propose supplementary types of display (e.g. gauge, ...).

### Interface Function 2

When enabled, Interface Function 2 adapts the P&ID GUI to show if the mandatory expected pump has been paired and supplements the P&ID GUI with control icons and displays of operating parameters of the paired pump.

The capability Interface Function 2 has the following description in the Device Shape file 20 of the mixer: Domain: “Control”, Purpose: “Pumping”, Role: “Consumer”, Restriction/Condition: exclusivity, mandatory, confirmed by operator. List of properties:

| | |
|---|---|
| Property | Attributes |
| Control_application | mandatory=no, type=text, access=OpcUa, value=″Pump_on_inlet1″ |
| Pump_Set_Start_Stop | mandatory=yes, type= boolean, access=OpcUa |
| Pump_Started | mandatory=yes, type= boolean, access=OpcUa |
| Pump_Set_Speed | mandatory=no, type= decimal, access=OpcUa |
| Pump_Speed_Value | mandatory=no, type= decimal, access=OpcUa |
| Pump_Speed_Unit | mandatory=no, type= text, access=OpcUa |
| Pump_Speed_significant _decimal_digits | mandatory=no, type= decimal, access=OpcUa |

This capability description means: the mixer, as a capability consumer with control skills, requires a system device which is a pump to be mandatorily paired to achieve the role defined for the pump connected to the inlet 1. To be paired, the system device will mandatorily have to make available a Start/Stop command and provide its current started status. The mixer will be able to control and monitor the pump speed if provided by the paired system device. The confirmation by the operator is required during the pairing procedure. Once paired, the mixer will have the exclusive usage of the system device which is a pump.

In a variant, the list of properties further includes at least one of:

| | |
|---|---|
| Property | Attributes |
| pump_speed_min_range | mandatory=no, type= decimal, access=OpcUa |
| pump_speed_max_range | mandatory=no, type= decimal, access=OpcUa |

In such a variant, the capability description further means that the mixer will be able to display the minimum and maximum values of a speed range if provided by the paired pump, in order to guide the operator when setting the pump speed.

### Interface Function 3

When enabled, Interface Function 3 adapts the P&ID GUI to show if the planed optional pump has been paired and supplements the P&ID GUI with control icons and displays of operating parameters of the paired pump.

The capability Interface Function 3 has the following description in the Device Shape file 20 of the mixer: Domain: “Control”, Purpose: “Pumping”, Role: “Consumer”, Restriction/Condition: exclusivity, optional, confirmed by operator. List of properties:

| | |
|---|---|
| Property | Attributes |
| Control_application | mandatory=no, type=text, access=OpcUa, value=″Pump_on_inlet2″ |
| Pump_Set_Start_Stop | mandatory=yes, type=Boolean, access=OpcUa |
| Pump_Started | mandatory=yes, type=Boolean, access=OpcUa |
| Pump_Set_Speed | mandatory=no, type= decimal, access=OpcUa |
| Pump_Speed_Value | mandatory=no, type=decimal, access=OpcUa |
| Pump_Speed_Unit | mandatory=no, type=text, access=OpcUa |
| Pump_Speed_significant _decimal_digits | mandatory=no, type= decimal, access=OpcUa |

This capability description means: the mixer, as a capability consumer with control skills, is able to control an optional system device that is a pump to achieve the predefined role for the pump connected to the inlet 2. To be paired, the system device will mandatorily have to make available a Start/Stop command and provide its current started status. The mixer will be able to control and monitor the pump speed if provided by the paired system device. The confirmation by the operator is required during the pairing procedure. Once paired, the mixer will have the exclusive usage of the system device which is a pump.

In a variant, the list of properties further includes at least one of:

| | |
|---|---|
| Property | Attributes |
| pump_speed_min_range | mandatory=no, type= decimal, access=OpcUa |
| pump_speed_max_range | mandatory=no, type= decimal, access=OpcUa |

In such a variant, the capability description further means that the mixer will be able to display the minimum and maximum values of a speed range if provided by the paired pump, in order to guide the operator when setting the pump speed.

### Interface Function 4

When enabled, Interface Function 4 adapts the P&ID GUI to show any other optional paired pump and supplements the P&ID GUI with control icons and displays of operating parameters of the paired pump.

The capability Interface Function 4 has the following description in the Device Shape file 20 of the mixer: Domain: “Control”, Purpose: “Pumping”, Role: “Consumer”, Restriction/Condition: exclusivity, optional. List of properties:

| | |
|---|---|
| Property | Attributes |
| Pump_Set_Start_Stop | mandatory=no, type=boolean, access=OpcUa |
| Pump_Started | mandatory=no, type=boolean, access=OpcUa |
| Pump_Set_Speed | mandatory=no, type=decimal, access=OpcUa |
| Pump_Speed_Value | mandatory=no, type=decimal, access=OpcUa |
| Pump_Speed_Unit | mandatory=no, type=text, access=OpcUa |
| Pump_Speed_significant _decimal_digits | mandatory=no, type=decimal, access=OpcUa |

This capability description means: the mixer, as a capability consumer with control skills, is able to control any other optional system device which is a pump. No mandatory properties are expected. The mixer will be able to start/stop the pump and to control and monitor the pump speed if provided by the paired system device. No confirmation by the operator is required during the pairing procedure. Once paired, the mixer will have the exclusive usage of the system device which is a pump.

It should be noted that not all interface functions of the GUI manager 17 of the mixer have been described here. The interface functions regarding the equipment modules in the mixer (agitator, controls of inlet valves, ...) are not described here. Only interface functions disabled when the mixer is not paired with the appropriate system device and enabled when the mixer is paired with the appropriate system device are described and at least other such interface function may be included in the GUI manager 17 of the mixer.

It should be further noted that the capabilities Interface Function 2, Interface Function 3 and Interface Function 4 illustrate three levels of capabilities that can become provided capabilities, namely predefined and mandatory capability such as Interface Function 2, predefined and optional capability such as Interface Function 3 and optional supplementary capability such as Interface Function 4.

### Description of the pH Sensor

The pH sensor includes a probe for sensing the pH of the biotechnological fluid. The probe forms a biotechnological fluid treater helper 21.

The pH sensor includes digital processing units including a microprocessor and/or a microcontroller, a memory and network connectivity.

The processing units are configured for controlling and monitoring the probe for sensing the flow, to which they are electrically wired.

On the processing units is installed a software package and stored a file 26 called Device Shape.

The processing units, the installed software package and the stored Device Shape file 26 form a digital controller 22.

The installed software package has the same architecture as the software package installed in the mixer, the software package installed in the pH sensor including: a DNP manager 25; a MtoM communication tool 24 having here an OPC UA server and an OPC UA client to support data exchanges with other system devices; and a GUI manager 23 that allows to display a GUI remotely on a device having an interactive screen, for e.g., a tablet or smartphone.

The file 26 called Device Shape contains a description of one interface function which is a consumed capability when in modified form.

### Description of the Capability Which Can Be Utilized by the Software Package of the pH Sensor:

When in modified form the single interface function described in the Device Shape file of the pH sensor keeps the display of the current value measured by pH sensor and a trend curve representing the variation of the pH in time and adds an indication that the pH sensor is paired with an appropriate other system device.

This capability has the following description in the Device Shape file 26 of the pH sensor: Domain: “Graphics”, Purpose: “Process Value Display”, Role: “Provider”, Restriction/Condition: none. List of properties:

| | |
|---|---|
| Property | Attributes |
| process_value_dim | mandatory=no, type=text, value=″pH″ |
| process_value | mandatory=no, type=decimal, access=OpcUa, tag=opc.tcp://pH/4:control/4:V,...) |
| process_value_name | mandatory=no, type=text, access=OpcUa, tag=opc.tcp://pH/4:control/4:name,...) |
| process_value_unit | mandatory=no, type=text, access=OpcUa, tag=opc.tcp://pH/4:control/4:unit,...) |

This capability description means: the pH sensor, as a capability provider, is able to provide a pH (and only a pH) process value display dataset to paired system devices using the OPC UA standard. The dataset includes a process value, its name and its unit. The OPC UA tag values for each data item is specified allowing the paired system devices to read these values with an OPC UA client. No specific restriction or condition is imposed for the negotiation or the pairing.

In a variant, the dataset further includes at least one of the minimum value or the maximum value of a range of values within which the pH process value is expected to be found.

### Description of the Flow Sensor

The flow sensor includes a probe for sensing the flow of biotechnological fluid. The probe forms a biotechnological fluid treater helper 21.

The flow sensor includes digital processing units including a microprocessor and/or a microcontroller, a memory, and network connectivity.

The processing units are configured for controlling and monitoring the probe for sensing the flow, to which they are electrically wired.

On the processing units is installed a software package and stored a file 26 called Device Shape.

The processing units, the installed software package and the stored Device Shape file 26 form a digital controller 22.

The installed software package has the same architecture as the software package installed in the mixer, the software package installed in the flow sensor including: a DNP manager 25; a MtoM communication tool 24 having here an OPC UA server and an OPC UA client to support data exchanges with other system devices; and a GUI manager 23 that allows to display a GUI remotely on a device having an interactive screen, for e.g., a tablet or smartphone.

The file 26 called Device Shape contains a description of one interface function which is a consumed capability when in modified form.

### Description of the Capability Which Can Be Utilized by the Software Package of the Flow Sensor

When in modified form the single interface function described in the Device Shape of the flow sensor replaces the display of the current value measured by the flow sensor and a trend curve representing the variation of the flow in time by the display of an indication that the flow sensor is paired with an appropriate other system device.

This capability has the following description in the Device Shape file 26 of the flow sensor: Domain: “Graphics”, Purpose: “Process Value Display”, Role: “Provider”, Restriction/Condition: none. List of properties:

| | |
|---|---|
| Property | Attributes |
| process_value_dim | mandatory=no, type=text, value=″Flow″ |
| process_value | mandatory=no, type=decimal, access=OpcUa, tag=opc.tcp://flow/4:control/4:V,...) |
| process_value_name | mandatory=no, type=text, access=OpcUa tag=opc.tcp://flow/4:control/4:name,...) |
| process_value_unit | mandatory=no, type=text, access=OpcUa, tag=opc.tcp://flow/4:control/4:unit,...) |

This capability description means: the flow sensor, as a capability provider, is able to provide a flow (and only a flow) process value display dataset to paired system devices using the OPC UA standard. The dataset includes a process value, its name and its unit. The OPC UA tag values for each of the data is specified allowing the paired system devices to read these values with an OPC UA client. No specific restriction or condition is imposed for the negotiation or the pairing.

In a variant, the dataset further includes at least one of the minimum value or the maximum value of a range of values within which the flow process value is expected to be found.

### Description of the Pump

The pump includes members for acting on the fluid for driving it, for instance the rollers of a peristaltic pump, and a motor for driving such members. The driving motor and the driven members acting on the fluid form a biotechnological fluid treater helper 21.

The pump includes digital processing units including a microprocessor and/or a microcontroller, a memory and network connectivity.

The processing units are configured for controlling and monitoring the motor, to which they are electrically wired.

On the processing units is installed a software package and stored a file 26 called Device Shape.

The processing units, the installed software package and the stored Device Shape file 26 form a digital controller 22.

The installed software package has the same architecture as the software package installed in the mixer: the software package installed in the pump includes: a DNP manager 25; a MtoM communication tool 24 having here an OPC UA server and an OPC UA client to support data exchanges with other system devices; and a GUI manager 23 that allows to display a GUI remotely on a device having an interactive screen, for e.g., a tablet or smartphone.

The file called Device Shape contains a description of an interface function which is a provided capability when enabled (Interface Function 1) and a description of an interface function which is a consumed capability when in modified form (Interface Function 2).

### Description of the Capabilities Which Can Be Utilized by the Software Package Of the Pump

As just mentioned, there are two such capabilities, respectively Interface Function 1 and Interface Function 2.

### Interface Function 1

When enabled, Interface Function 1 supplements the GUI with data provided by a paired system device, whatever the paired system device is.

The capability Interface Function 1 has the following description in the Device Shape file 26 of the pump: Domain: “Graphics”, Purpose: “Process Value Display”, Role: “Consumer”, Restriction/Condition: max=1, optional. List of properties:

| | |
|---|---|
| Property | Attributes |
| process_value_dim | mandatory=yes, type=text, value=″Flow″ |
| process_value | mandatory=yes, type=decimal, access=OpcUa |
| process_value_name | mandatory=yes, type=text, access=OpcUa |
| process_value_unit | mandatory=yes, type=text, access=OpcUa |

This capability description means: the pump, as a capability consumer with graphic skills, is able to optionally display one and only one flow process value if the paired system device provides at least the process value, the process value name and unit using the OPC UA standard. No other restriction or condition is imposed for the negotiation or pairing except the maximum number of authorized pairing.

In a variant, the list of properties further includes at least one of:

| | |
|---|---|
| Property | Attributes |
| process_value_min_range | mandatory=no, type=no, access=OpcUa |
| process_value_max_range | mandatory=no, type=no, access=OpcUa |

In such a variant, the capability description further means that the paired system devices may optionally provide the minimum and the maximum values of a range associated to the flow process value.

### Interface Function 2

When in original form, Interface Function 2 displays the pump motor speed and has a control icon allowing to start/stop the motor of the pump and a control icon allowing to set the pump motor speed. When in modified form, the two control icons are removed from the GUI, only the display of the pump motor speed is kept on display.

The capability Interface Function 2 has the following description in the Device Shape file of the pump: Domain: “Control”, Purpose: “Pumping”, Role: “Provider”, Restriction/Condition: exclusivity. List of properties:

| | |
|---|---|
| Property | Attributes |
| Pump_Set_Start_Stop | mandatory=no, type=boolean, access=OpcUa, tag=opc.tcp://start |
| Pump_Started | mandatory=no, type=boolean, access=OpcUa, tag=opc.tcp://started |
| Pump_Set_Speed | mandatory=no, type=decimal, access=OpcUa, tag=opc.tcp://speed/4:V |
| Pump_Speed_Value | mandatory=no, type=decimal, access=OpcUa, tag=opc.tcp://speed/4:S |
| Pump_Speed_Unit | mandatory=no, type=text, access=OpcUa, tag=opc.tcp://speed/4:U |
| Pump_Speed_significant _decimal_digits | mandatory=no, type=decimal, access=OpcUa, tag=opc.tcp://speed/4:d |

This capability description means: the pump 13, as a capability provider with pumping skills, is able to provide the control and monitoring on its pumping function using the OPC UA standard. The paired system device will have the exclusivity of the usage of the pumping function and will be able to start/stop the pump, to set the pump speed and to retrieve the current pumping status and speed. The OPC UA tag values for each of the control and monitoring are specified allowing the consumer to use the pump with an OPC UA client.

In a variant, the list of properties further includes at least one of:

| | |
|---|---|
| Property | Attributes |
| pump_speed_min_range | mandatory=no, type= decimal, access=OpcUa |
| pump_speed_max_range | mandatory=no, type= decimal, access=OpcUa |

In such a variant, the capability description further means that the pump 13 will be able to provide the minimum and maximum values of a speed range.

### Discovery, Negotiation, Pairing (DNP)

A description will now be given on how system devices 13 and 14 can cooperate over the network 12 for establishing a paired condition.

This is mainly carried out by the DNP managers 19 and 25 in the system devices 13 and 14, through steps of discovery, negotiation and pairing, described below.

### Discovery

When connected to the network with IP (e.g. ethernet, Wi-Fi, Bluetooth or cellular such as 5G), the system device can see and can be seen by another connected system device that includes a discovery tool.

Several architectures already exist that enables discovery across a network (e.g. The ‘Bonjour’ protocol defined by Apple, and here global or local discovery proposed by the OPC UA standard).

The visibility is only restrained by potential security policies in place on the network

The discovery tool allows a system device to maintain an up-to-date list of visible system devices it can exchange information with. This list is notably updated when a new system device is connected to or disconnected from the network.

### Neaotiation

Based on the capability descriptions provided in the Device Shape files, a negotiation starts between the connected system devices wherein each system device on the network: consults the capability descriptions exhibited by the other system devices, identifies matching capabilities based on the capability features Domain, Purpose and Role, verifies that it can respect the restrictions and conditions associated with the matching capabilities, and checks if the list of properties exhibited with the matching capabilities are the expected ones.

The Negotiation procedure occurs each time a new system device is discovered on the network, disconnected from the network, or no longer reachable.

### Pairing

Once the negotiation is achieved, the different contributors have agreed on how to adapt themselves to respect the restrictions and the conditions as expressed for each of the shared capabilities.

The pairing will complete the procedure, confirming the negotiation between two system devices.

The capability consumer (respectively the provider) memorizes the identification and location - here OPC UA endpoint - of the provider (respectively the consumer) to enable later data exchange.

Both the capability consumer and the capability provider apply restrictions and conditions - if some - agreed during the negotiation.

The capability consumer locally publishes the access to the list of properties in the Device Shape file of the consumed capability, so that the GUI manager installed in the capability consumer can exchange data with the paired capability provider.

This can be achieved, for example, using a standard Publication/Subscription approach: when the system device is powered on, a specific data queue is created by the DNP manager for each different pair (domain, purpose) described in the Device Shape file; the GUI manager subscribe to the (domain, purpose) queues it is interested in; each time a pairing occurs, the DNP manager publishes the access information in the corresponding (domain, purpose) data queue; the GUI manager subscriber to this queue will automatically be triggered and will access to the description and will accordingly adapt.

Specific situations could occur where different system devices on the network can provide a specific capability expected by a consumer. In such a case, the pairing might require a decision by a human operator to manually select the capability provider.

A similar situation requiring the approval from the operator occurs when this is explicitly expressed in the capability description as a condition.

It should be noted that the pairing removal requires the intervention of an operator to be able to distinguish between an intentional disconnection of a system device, and a communication failure.

Without this voluntary action from the user, any disconnection of a system device is considered as an anomaly and processed accordingly such as generating an alarm.

A description of pairing removal will be given now.

### Pairing Removal

As just mentioned, the pairing removal of a system device from another system device to which it is paired requires a voluntary and explicit action of the user, so as to enable to distinguish between an intentional disconnection of a system device and a communication failure.

This is carried out for instance by providing a dedicated menu accessible to the user on the graphical user interface of a system device, such menu listing each other system device with which the system device is paired, and from such menu the user can explicitly request to remove the pairing with a system device selected in the list of the menu.

When the user requests to remove the pairing with a selected other system device, steps are carried out (i) for removing the effects of the step of pairing, (ii) for removing the effects, if any, of the step of negotiation and (iii) for temporarily preventing the system device and the selected other system device to carry out a negotiation step.

For removing the effects of the step of pairing, the DNP manager 19 or 25 of the system device locally publishes the status change of each capability consumed from or provided to the selected other system device and sends to the selected other system device through the MtoM communication tool 18 or 24 a request to proceed to pairing removal. The DNP manager 19 or 25 of the selected other system device in turn locally publishes the status change of each capability consumed from or provided to the system device and sends to the system device through the MtoM communication tool 18 or 24 an acknowledgement receipt of the request to proceed to pairing removal.

In the system device and in the selected other system device, the GUI manager 17 or 23 is warned of the status change of each concerned capability and adapts accordingly.

For removing the effects of the step of negotiation, if any, namely if the capability consumer and the capability provider have applied restrictions and conditions agreed during the negotiation (e.g. exclusivity), these restrictions and conditions are invalidated.

For temporarily preventing the system device and the selected other system device to carry out a negotiation step, because the capabilities previously shared are now again available for negotiation, a quarantine is implemented for instance using a timeout such as not accepting the concerned system device in a negotiation step during a predetermined duration, the length of which is not really important but may, for example, be one minute, or 2 minutes, or 3 minutes, or 4 minutes, or 5 minutes, or 10 minutes, or using network connectivity such as ignoring the concerned system device until it is disconnected from the network and reconnected.

A detailed description will now be given of the pairing of the mixer and the pH sensor, of the pairing of the pump and the flow sensor, of the pairing of the previously paired mixer and pH sensor with the previously paired pump and flow sensor, and of the removal from the mixer of the paired pump and flow sensor while the pH sensor remains paired with the mixer.

### Pairing of the Mixer and the pH Sensor

### Features of the Mixer GUI

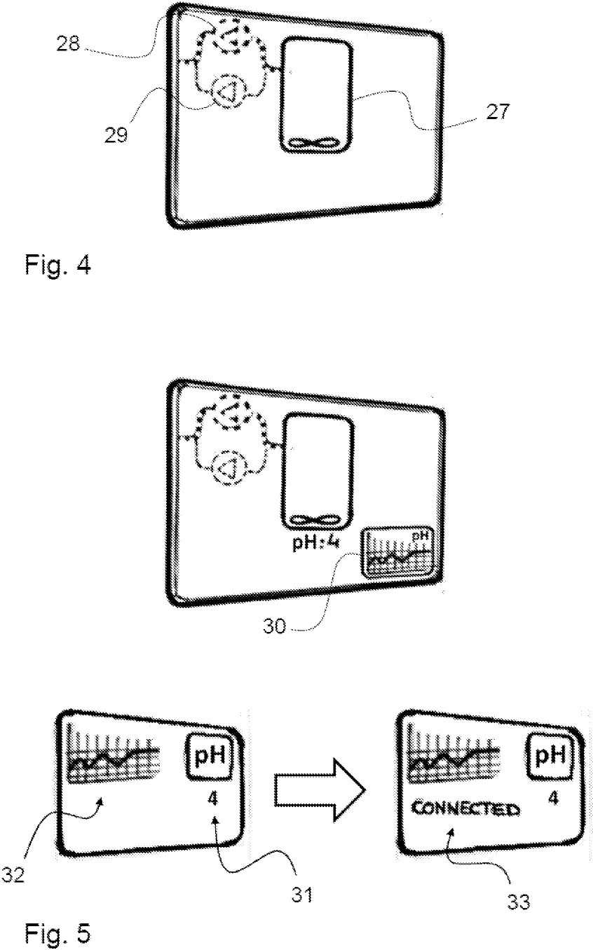

When the operator powers the mixer on, he can access to a basic P&ID GUI shown on FIG. 4, locally on an interactive screen or remotely on a device having an interactive screen, for e.g., a tablet or smartphone.

The basic P&ID GUI represents the different components required for the mixing process, an icon 27 representing a tank provided with an agitator, an icon 28 representing a mandatory pump in fluidic communication with inlet 1 of the tank and an icon 29 representing an optional pump in fluidic communication with inlet 2 of the tank.

In the basic P&ID GUI, icon 27 is displayed in a way showing that the tank provided with an agitator is present and operational (for instance displayed in permanent solid lines), icon 28 is displayed in a way showing that the mandatory pump is missing (for instance displayed in blinking phantom lines) and icon 29 is displayed in a way showing that the optional pump is missing (for instance displayed in permanent phantom lines).

The way the two icons 28 and 29 representing a pump are displayed is dependent on the pairing context and is automatically updated for showing that a corresponding pump system device is paired (for instance by then displaying the icon in permanent solid lines).

A further description of the P&ID GUI as regards the pairing with pumps will be given later in the section relating to the pairing of the mixer with the pump. Now will be given a description of the P&ID GUI as regards the pairing with the pH sensor.

When the mixer is powered on, its DNP manager creates in the digital controller of the mixer a (“Graphics”, “Process Value Display”) capability data queue. The GUI manager of the pump subscribes to the (“Graphics”, “Process Value Display”) capability data queue and displays the basic P&ID GUI until a new capability description is published in this queue.

When later a system device providing a (“Graphics”, “Process Value Display”) capability with the expected properties is paired with the mixer, the DNP manager of the mixer publishes the description of this capability in the (“Graphics”, “Process Value Display”) data queue, the GUI manager is triggered, accesses the published description and accordingly adapts the P&ID GUI, as shown on FIG. 5 at the top, by further displaying the current process value (here the pH) and a trend curve 30 for the process value.

Indeed, as described above, the Device Shape file of the mixer includes a capability named Interface Function 1 which, when enabled, supplements the P&ID GUI with process data provided by paired system devices, whatever the paired system devices are; and this capability has a description meaning: the mixer, as a capability consumer with graphic skills, is able to display a process value from several paired system devices if these paired system devices provide at least the process value, the process value name and unit and optionally the number of significant decimal digits using the OPC UA standard. No restriction or condition are imposed for the negotiation or pairing.

### Features of the pH Sensor GUI

When the operator powers the pH sensor on, he can access to an original GUI, shown on FIG. 5 at the bottom left, remotely on a device having an interactive screen, for e.g., a tablet or smartphone.

The original GUI includes the current value 31 measured by the pH sensor and a trend curve 32 representing the variation of the pH in time.

When the pH sensor is powered on, its DNP manager creates in the digital controller of the pH sensor a (“Graphics”, “Process Value Display”) capability data queue. The GUI manager of the pH sensor subscribes to the (“Graphics”, “Process Value Display”) capability data queue and displays the original GUI until a new capability description is published in this queue.

When later a system device consuming a (“Graphics”, “Process Value Display”) capability with the expected properties is paired with the pH sensor, the DNP manager of the pH sensor publishes the description of this capability in the (“Graphics”, “Process Value Display”) data queue, the GUI manager is triggered, accesses the published description and accordingly adapts the GUI, as shown on FIG. 5 at the bottom right, by additionally displaying the message “CONNECTED” 33.

Indeed, as described above, the Device Shape file of the pH sensor includes a capability which, when in modified form, keeps the display of the current value measured by the pH sensor and a trend curve representing the variation of the pH in time and adds an indication that the flow sensor is paired with an appropriate other system device, this indication being here the message “CONNECTED” 33; and this capability has a description meaning: the pH sensor, as a capability provider, is able to provide a pH (and only a pH) process value display dataset to paired system devices using the OPC UA standard. The dataset includes a process value, its name and its unit. The OPC UA tag values for each data item is specified allowing the paired system device to read these values with an OPC UA client. No specific restriction or condition is imposed for the negotiation or the pairing.

### DNP Sequence

The operator connects the mixer and the pH sensor to the same network 12.

DNP as described above is carried out and when done the P&ID GUI of the mixer and the GUI of the pH sensor are automatically updated: the P&ID GUI of the mixer is further displaying the current pH value and a trend curve 30 for the pH value; and the GUI of the pH sensor additionally displays a message “CONNECTED” 33.

It goes without saying that for enabling the pH sensor to sense the pH of the fluid being treated by the mixer, the pH sensor must be physically to the mixer.

The message “CONNECTED” on the GUI of the pH sensor clearly shows that the pH sensor is not in stand-alone condition but in paired condition.

A detailed description of an example of DNP sequence will now be given.

In this example the pH sensor is first connected to the network 12 with IP, but of course the reverse is possible.

Since the pH sensor is a capability provider for the capability described in its Device Shape file 26, as long as the pH sensor is connected to the network 12, its MtoM communication tool 24 is provided in real time with the sensed pH value and makes it available on the network 12 thanks to the OPC UA server it includes, at the OPC UA endpoint given in the capability description in the Device Shape file 26, namely opc.tcp://pH/4:control/4:

For enabling DNP, in the pH sensor the DNP manager 25 provides the MtoM communication tool 24 with data to make available the capability description in the Device Shape file 26, including the properties in the capability description; and the DNP manager 25 creates in the digital controller 22 of the pH sensor a (“Graphics”, “Process Value Display”) data queue for this capability. The MtoM communication tool 24 then waits the discovery of another system device on the network 12.

Still in the pH sensor, for preparing to adapt, the GUI manager 23 subscribes to the (“Graphics”, “Process Value Display”) data queue created by the DNP manager 25 and displays the original form of the GUI.

The mixer in turn connects to the network and carries out similar steps in accordance with its Device Shape file 20, as detailed below.

For enabling DNP, in the mixer the DNP manager 19 provides the MtoM communication tool 18 with data to make available the capability descriptions in the Device Shape file 20, namely Interface Function 1, Interface Function 2, Interface Function 3 and Interface Function 4, including the properties in each capability description; and the DNP manager 19 creates in the digital controller 16 of the mixer a (“Graphics”, “Process Value Display”) data queue for the capability Interface Function 1 and a (“Control”, “Pumping”) data queue for the capabilities Interface Function 2, Interface Function 3 and Interface Function 4. The MtoM communication tool 18 then waits the discovery of another system device on the network.

Still in the mixer, for preparing to adapt, the GUI manager 17 subscribes to the (“Graphics”, “Process Value Display”) and (“Control”, “Pumping”) data queues created by the DNP manager and displays the basic P&ID GUI.

In the pH sensor, when the MtoM communication tool 24 discovers that the mixer is connected to the network 12, it informs of this discovery the DNP manager 25 which then requests the MtoM communication tool 24 to provide the capability descriptions exhibited by the mixer. When provided, the capability descriptions are reviewed by the DNP manager 25 which identifies a matching between the capability Interface Function 1 made available by the mixer and the local capability with the restrictions/conditions applicable. The DNP manager 25 then requests the MtoM communication tool 24 to propose to the mixer to apply pairing between the local capability and the capability Interface Function 1 in the mixer. When the MtoM communication tool 24 receives the pairing acceptance it provides the pairing acceptance to the DNP manager 25 which publishes the description of the capability Interface Function 1 of the mixer in the (“Graphics”, “Process Value Display”) data queue and requests the MtoM communication tool 24 to confirm application for the capability Interface Function 1 of the mixer. The GUI manager 23 is automatically notified of the publication in the (“Graphics”, “Process Value Display”) data queue and receives the description of the capability Interface Function 1 of the mixer and adapts accordingly, namely displays the modified form of the GUI, i.e. additionally displays the message “CONNECTED” 33. The modified form of the GUI is displayed until pairing removal occurs.

In the mixer, when the MtoM communication tool 18 discovers that the pH sensor is connected to the network 12, it informs of this discovery the DNP manager 19 which then requests the MtoM communication tool 18 to provide the capability descriptions exhibited by the pH sensor. When provided, the capability descriptions are reviewed by the DNP manager 19 which identifies a matching between the capability Interface Function 1 made available by the pH sensor and the local capability with the restrictions/conditions applicable. When the MtoM communication tool 18 receives from the pH sensor the confirmation of application for the capability Interface Function 1, the confirmation is transferred to the DNP manager 19 which publishes the description of the capability of the pH sensor in the (“Graphics”, “Process Value Display”) data queue. The GUI manager 17 is automatically notified of the publication in the (“Graphics”, “Process Value Display”) data queue and receives the description of the capability of the pH sensor, including the OPC UA tag for the flow value opc.tcp://pH/4:control/4:V, and adapts accordingly, namely adapts the P&ID GUI by further displaying the current pH value and a trend curve 30 for the pH value, the tag provided for the pH value being used, thanks to the OPC UA client in the MtoM communication tool 18, for continuously update the pH value until pairing removal occurs.

### Pairing of the Pump and the Flow Sensor

### Features of the Pump GUI

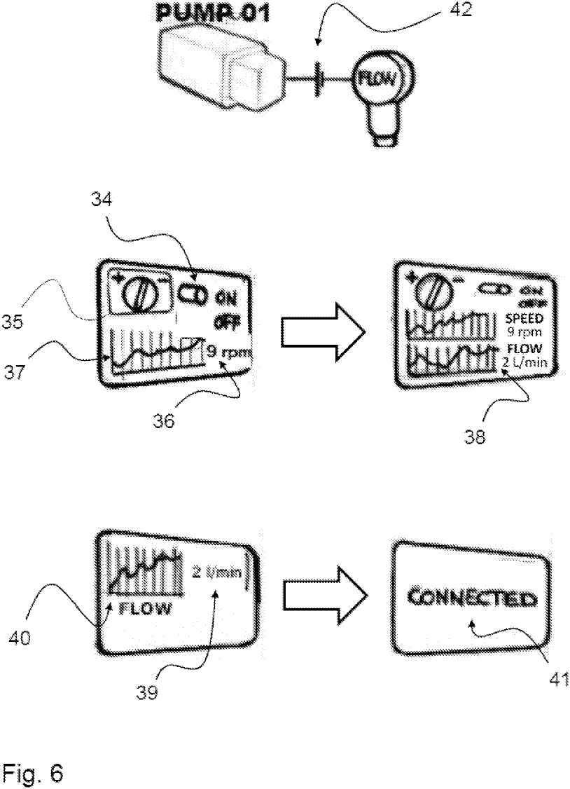

When the operator powers the pump on, he can access to a basic GUI, shown on FIG. 6 at the middle left, remotely on a device having an interactive screen, for e.g., a tablet or smartphone.

The basic P&ID GUI includes a Start/Stop button 34 allowing to operate the pump, a variator 35 allowing to modify the pump speed, a display of the current pump speed 36 and a display of a curve 37 representing the variation of the pump speed in time.

When the pump is powered on, its DNP manager 25 creates in the digital controller 22 of the pump a (“Graphics”, “Process Value Display”) capability data queue. The GUI manager 23 of the pump subscribes to the (“Graphics”, “Process Value Display”) capability data queue and displays the basic GUI until a new capability description is published in this queue.

When later a system device providing a (“Graphics”, “Process Value Display”) capability with the expected properties is paired with the pump, the DNP manager 25 of the pump publishes the description of this capability in the (“Graphics”, “Process Value Display”) data queue, the GUI manager 23 is triggered, accesses the published description and accordingly adapts the GUI, as shown on FIG. 6 at the middle right, by further displaying the current flow value and a trend curve 38 for the flow value.

Indeed, as described above, the Device Shape file of the pump includes a capability named Interface Function 1 which, when enabled, supplements the GUI with data provided by a paired system device; and this capability has a description meaning: the pump, as a capability consumer with graphic skills, is able to optionally display one and only one flow process value if the paired system device provides at least the process value, the process value name and unit using the OPC UA standard. No other restriction or condition is imposed for the negotiation or pairing except the maximum number of authorized pairing.

### Features of the Flow Sensor GUI

When the operator powers the flow sensor on, he can access to an original GUI, shown on FIG. 6 at the bottom left, remotely on a device having an interactive screen, for e.g., a tablet or smartphone.

The original GUI includes a display of the current value 39 measured by the flow sensor and a display of a trend curve 40 representing the variation of the flow in time.

When the flow sensor is powered on, its DNP manager 25 creates in the digital controller 22 of the flow sensor a (“Graphics”, “Process Value Display”) capability data queue. The GUI manager 23 of the flow sensor subscribes to the (“Graphics”, “Process Value Display”) capability data queue and displays the original GUI until a new capability description is published in this queue.

When later a system device consuming a (“Graphics”, “Process Value Display”) capability with the expected properties is paired with the flow sensor, the DNP manager 25 of the flow sensor publishes the description of this capability in the (“Graphics”, “Process Value Display”) data queue, the GUI manager 23 is triggered, accesses the published description and accordingly adapts the GUI, as shown on FIG. 6 at the bottom right, by only displaying the message “CONNECTED” 41.

Indeed, as described above, the Device Shape file 26 of the flow sensor includes a capability which, when in modified form, replaces the display of the current value measured by the flow sensor and the display of a trend curve representing the variation of the flow in time by the display of an indication that the flow sensor is paired with an appropriate other system device, this indication being here the message “CONNECTED” 41; and this capability has a description meaning: the flow sensor, as a capability provider, is able to provide a flow (and only a flow) process value display dataset to paired system devices using the OPC UA standard. The dataset includes a process value, its name and its unit. The OPC UA tag values for each of the data is specified allowing the paired system devices to read these values with an OPC UA client. No specific restriction or condition is imposed for the negotiation or the pairing.

### DNP Sequence

The operator connects the pump and the flow sensor to the same network 12 (FIG. 3).

DNP as described above is carried out and when done the GUI of the pump and the GUI of the flow sensor are automatically updated: the P&ID GUI of the pump is further displaying the current flow value and a trend curve 38 for the flow value; and the GUI of the flow sensor only displays a message “CONNECTED” 41.

It goes without saying that for enabling the flow sensor to sense the flow of the fluid driven by the pump, the flow sensor must be physically coupled in a known manner to the pump or to pipes in which flows the fluid driven by the pump, as shown on FIG. 6 at the top by reference numeral 42.

It should be noted that since the pump and the flow sensor are both a machine helper 14, the physical coupling 42 is between two treater helpers 21 (and not between a fluid treater 15 and a treater helper 21).

It should be further noted that the DNP manager 25 of the pump is able to behave as the DNP manager 19 of a bioprocess machine 13 with respect to the DNP manager 25 of the flow sensor for cooperating over the network 12 for establishing a paired condition between the pump and the flow sensor, thanks to the fact that the capability Interface Function 1 of the pump has as Role feature “Consumer”, just as each capability in the Device Shape file 20 of a bioprocess machine 13.

The message “CONNECTED” 41 on the GUI of the flow sensor clearly shows that the flow sensor is not in stand-alone condition but in paired condition.

A detailed description of an example of DNP sequence will now be given.

In this example the flow sensor is first connected to the network 12 with IP, but of course the reverse is possible.

Since the flow sensor is a capability provider for the capability described in its Device Shape file 26, as long as the flow sensor is connected to the network 12, its MtoM communication tool 24 is provided in real time with the sensed flow value and makes it available on the network 12 thanks to the OPC UA server it includes, at the OPC UA endpoint given in the capability description in the Device Shape file 26, namely opc.tcp://flow/4:control/4:

For enabling DNP, in the flow sensor the DNP manager 25 provides the MtoM communication tool 24 with data to make available the capability description in the Device Shape file 26, including the properties in the capability description; and the DNP manager 25 creates in the digital controller 22 of the flow sensor a (“Graphics”, “Process Value Display”) data queue for this capability. The MtoM communication tool 24 then waits the discovery of another system device on the network 12.

Still in the flow sensor, for preparing to adapt, the GUI manager 23 subscribes to the (“Graphics”, “Process Value Display”) data queue created by the DNP manager 25 and displays the original form of the GUI.

The pump in turn connects to the network and carries out similar steps in accordance with its device Shape file 26, as detailed below.

As regards the capability Interface Function 2 (for which the pump is a capability provider), as long as the pump is connected to the network 12, its MtoM communication tool 24 is provided in real time with the operating parameters of the pump (Start/Stop control, started status, speed setting and value) and makes it available on the network 12 thanks to the OPC UA server it includes, at the OPC UA endpoints given in the capability description in the Device Shape file 26, respectively opc.tcp://start, opc.tcp://started and opc.tcp://speed/4:

For enabling DNP, in the pump the DNP manager 25 provides the MtoM communication tool 24 with data to make available the capability descriptions in the Device Shape file 26, namely Interface Function 1 and Interface Function 2, including the properties in each capability description; and the DNP manager 25 creates in the digital controller 22 of the pump a (“Graphics”, “Process Value Display”) data queue for the capability Interface Function 1 and a (“Control”, “Pumping”) data queue for the capability Interface Function 2. The MtoM communication tool 24 then waits the discovery of another system device on the network 12.

Still in the pump, for preparing to adapt, the GUI manager 23 subscribes to the (“Graphics”, “Process Value Display”) and (“Control”, “Pumping”) data queues created by the DNP manager 25 and displays the basic GUI.

In the flow sensor, when the MtoM communication tool 24 discovers that the pump is connected to the network 12, it informs of this discovery the DNP manager 25 which then requests the MtoM communication tool 24 to provide the capability descriptions exhibited by the pump. When provided, the capability descriptions are reviewed by the DNP manager 25 which identifies a matching between the capability Interface Function 1 exhibited by the pump and the local capability with the restrictions/conditions applicable. The DNP manager 25 then requests the MtoM communication tool 24 to propose to the pump to apply pairing between the local capability and the capability Interface Function 1 in the pump. When the MtoM communication tool 24 receives the pairing acceptance it provides the pairing acceptance to the DNP manager 25 which publishes the description of the capability Interface Function 1 of the pump in the (“Graphics”, “Process Value Display”) data queue and requests the MtoM communication tool 24 to confirm application for the capability Interface Function 1 of the pump. The GUI manager 23 is automatically notified of the publication in the (“Graphics”, “Process Value Display”) data queue and receives the description of the capability Interface Function 1 of the pump and adapts accordingly, namely displays the modified form of the GUI, i.e. only displays the message “CONNECTED” 41. The modified form of the GUI is displayed until pairing removal occurs.

In the pump, when the MtoM communication tool 24 discovers that the flow sensor is connected to the network 12, it informs of this discovery the DNP manager 25 which then requests the MtoM communication tool 24 to provide the capability descriptions exhibited by the flow sensor. When provided, the capability descriptions are reviewed by the DNP manager 25 which identifies a matching between the capability Interface Function 1 made available by the flow sensor and the local capability with the restrictions/conditions applicable. When the MtoM communication tool 24 receives from the flow sensor the confirmation of application for the capability Interface Function 1, the confirmation is transferred to the DNP manager 25 which publishes the description of the capability of the flow sensor in the (“Graphics”, “Process Value Display”) data queue. The GUI manager 23 is automatically notified of the publication in the (“Graphics”, “Process Value Display”) data queue and receives the description of the capability of the flow sensor, including the OPC UA tag for the flow value opc.tcp://flow/4:control/4:V, and adapts accordingly, namely adapts the GUI as shown on FIG. 6 at the middle right by further displaying the current flow value and a trend curve 38 for the flow value, the tag provided for the flow value being used, thanks to the OPC UA client in the MtoM communication tool 24, for continuously update the flow value until pairing removal occurs.

It should be noted that there is in the Device Shape file 26 of the pump a further capability enabling the pump to provide the flow values sensed by the flow sensor as if the flow sensor was part of the pump.

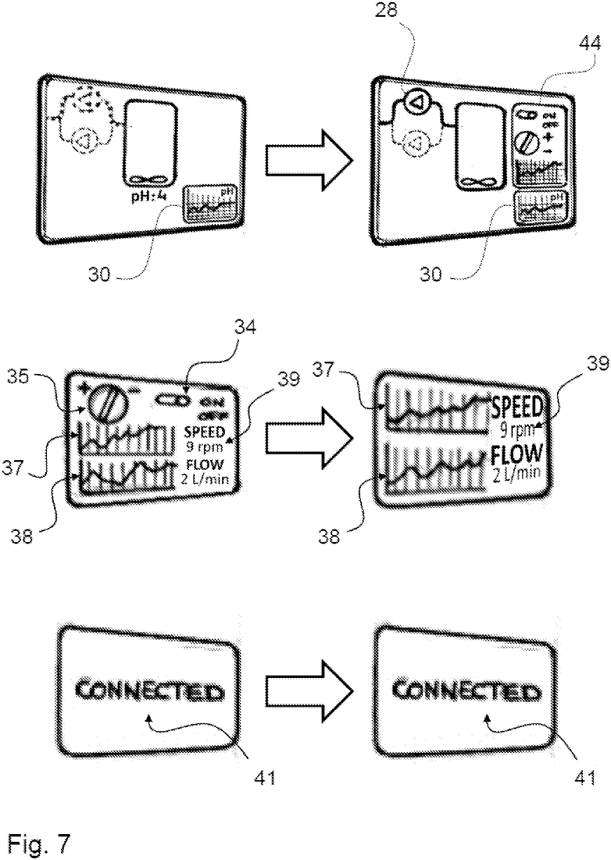

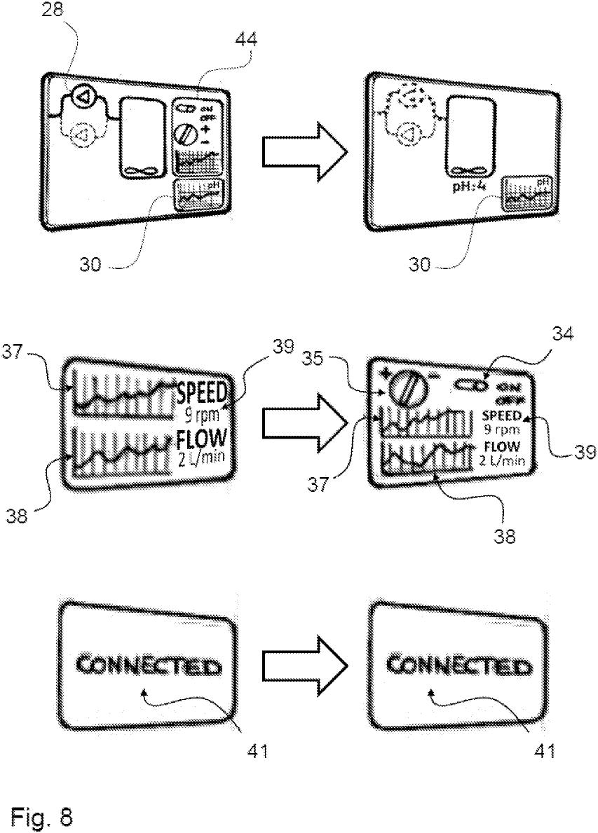

This will be described later. At the moment, it suffices to note that this enables the trend curve of the flow to be included in the control panel 44 in the P&ID GUI of the mixer, as shown at the top of FIGS. 7 and 8.

### Pairing of the Previously Paired Mixer and pH Sensor With the Previously Paired Pump and Flow Sensor

### Further Features of the Mixer GUI

As described above, upon pairing with the pH sensor the P&ID GUI of the mixer is supplemented, with respect to the basic P&ID, with a display of the pH value and a display of a trend curve for the pH value, as shown on FIG. 5 at the top.

As described above, the basic P&ID GUI, shown on FIG. 4, has an icon 27 representing a tank provided with an agitator, an icon 28 representing a mandatory pump in fluidic communication with inlet 1 of the tank and an icon 29 representing an optional pump in fluidic communication with inlet 2 of the tank; icon 27 is displayed in a way showing that the tank provided with an agitator is present and operational (for instance displayed in permanent solid lines), icon 28 is displayed in a way showing that the mandatory pump is missing (for instance displayed in blinking phantom lines) and icon 29 is displayed in a way showing that the optional pump is missing (for instance displayed in permanent phantom lines). The way the two icons 28 and 29 representing a pump are displayed is dependent on the pairing context and is automatically updated for showing that a corresponding pump system device is paired (for instance by then displaying the icon in permanent solid lines).

Further details as regards the pairing with the pH sensor are given elsewhere in the present description.

Further details will be given now as regards the pairing with pumps.

When the mixer is powered on, its DNP manager 19 creates in the digital controller 16 of the mixer a (“Control”, “Pumping”) capability data queue. The GUI manager 17 of the mixer subscribes to the (“Control”, “Pumping”) capability data queue. The icon 28 is displayed in a way showing that the mandatory pump is missing (for instance displayed in blinking phantom lines) until a new capability description is published in this queue with a Control_Local_Name equal to “Pump_on_inlet1”. The icon 29 is displayed in a way showing that the optional pump is missing (for instance displayed in permanent phantom lines) until a new capability description is published in this queue with a Control_Local_Name equal to “Pump_on_inlet2”.

When later a system device providing a (“Control”, “Pumping”) capability is paired with the mixer, the DNP manager 19 of the mixer publishes the description of this capability completed with Control_Local_Name equal to “Pump_on_inlet1” in the (“Control”, “Pumping”) capability data queue, the GUI manager 17 is triggered, accesses the published description and accordingly adapts the P&ID GUI by displaying the icon 28 in a way showing that the mandatory pump is paired (for instance displayed in permanent solid lines), as illustrated on FIG. 7 at the top right.

Indeed, as described above, the Device Shape file 20 of the mixer includes a capability named Interface Function 2 which, when enabled, adapts the P&ID GUI to show if the mandatory expected pump has been paired and supplements the P&ID GUI with control icons and displays of operating parameters of the paired pump; and this capability has a description meaning: the mixer, as a capability consumer with control skills, requires a system device which is a pump to be mandatorily paired to achieve the role defined for the pump connected to the inlet 1. To be paired, the system device will mandatorily have to make available a Start/Stop command and provide its current started status. The mixer will be able to control and monitor the pump speed if provided by the paired system device. The confirmation by the operator is required during the pairing procedure. Once paired, the mixer will have the exclusive usage of the system device which is a pump.

Similarly, when later a system device providing a (“Control”, “Pumping”) capability is paired with the mixer, the DNP manager 19 of the mixer publishes the description of this capability completed with Control_Local_Name equal to “Pump_on_inlet2” in the (“Control”, “Pumping”) capability data queue, the GUI manager 17 is triggered, accesses the published description and accordingly adapts the P&ID GUI by displaying the icon 29 in a way showing that the optional pump is paired (for instance displayed in permanent solid lines).

Indeed, as described above, the Device Shape file 20 of the mixer includes a capability named Interface Function 3 which, when enabled, adapts the P&ID GUI to show if the planed optional pump has been paired and supplements the P&ID GUI with control icons and displays of operating parameters of the paired pump; and this capability has a description meaning: the mixer, as a capability consumer with control skills, is able to control an optional system device which is a pump to achieve the predefined role for the pump connected to the inlet 2. To be paired, the system device will mandatorily have to make available a Start/Stop command and provide its current started status. The mixer will be able to control and monitor the pump speed if provided by the paired system device. The confirmation by the operator is required during the pairing procedure. Once paired, the mixer will have the exclusive usage of the system device which is a pump.

### Further Features of the Pump GUI

The operator now connects the pump on the same network 12.

When the mixer discovers the pump, the P&ID GUI of the mixer warns the operator (with a non-illustrated display, for instance in a pop-up window) that a pump that fulfills the requirements expected for a pump is available and can be used, and request to select one of the possible pumps.

Once the operator has physically connected the pump on the inlet 1 of the mixer, the choice inlet 1 may be selected.

The GUI of the flow sensor remains unchanged, that is to say still displays the message 41, as shown on FIG. 7 at the bottom.

The P&ID GUI of the mixer and the GUI of the pump are automatically updated.

In the P&ID GUI of the mixer, as shown on FIG. 7 at the top, icon 28 is displayed in a way showing that the mandatory pump on the inlet 1 is present and operational (for instance displayed in permanent solid lines), and a control panel 44 is now present on the P&ID GUI of the mixer, allowing the operator to control and monitor the pump on the inlet 1. The control panel 44 includes a Start/Stop button allowing to operate the pump, a variator allowing to modify the pump speed and a display of a trend curve representing the variation of the flow measured by the flow meter paired with the pump.

In the GUI of the pump, as shown on FIG. 7 at the middle, the Start/Stop button 34 allowing to operate the pump and the variator 35 allowing to modify the pump speed are removed. Only the display of the flow and the speed are kept.

When the pump is powered on, its DNP manager 25 creates in the digital controller 22 of the pump a (“Control”, “Pumping”) capability data queue. The GUI manager 23 of the pump subscribes to the (“Control”, “Pumping”) capability data queue and displays the basic GUI until a new capability description is published in this queue.

When later a system device providing a (“Control”, “Pumping”) capability with the expected properties is paired with the pump, the DNP manager 25 of the pump publishes the description of this capability in the (“Control”, “Pumping”) capability data queue, the GUI manager 23 is triggered, accesses the published description and accordingly adapts the GUI by removing the Start/Stop button 34 allowing to operate the pump and the variator 35 allowing to modify the pump speed.

Indeed, as described above, the Device Shape file 26 of the pump includes a capability named Interface Function 2 which, when in original form, displays the pump motor speed and has a control icon (button 34) allowing to start/stop the motor of the pump and a control icon (variator 35) allowing to set the pump motor speed. When in modified form, the two control icons are removed from the GUI, only the display of the pump motor speed is kept on display; and this capability has a description meaning: the pump, as a capability provider with pumping skills, is able to provide the control and monitoring on its pumping function using the OPC UA standard. The paired system device will have the exclusivity of the usage of the pumping function and will be able to start/stop the pump, to set the pump speed and to retrieve the current started status and speed. The OPC UA tag values for each of the control and monitoring are specified allowing the consumer to use the pump with an OPC UA client.

### DNP Sequence

The mixer (previously paired with the pH sensor) and the pump (previously paired with the flow sensor) are deployed on the same network 12, so that they can see each other and start the negotiation step.

The pump makes available the capability Interface Function 2.

The Mixer makes available three capabilities with the same domain and purpose (“Control”, “Pumping”): the capability Interface Function 2 requiring a pumping system to be mandatorily paired to achieve the role defined for the pump connected to the inlet 1; the capability Interface Function 3 enabling the mixer to control and monitor an optional pumping system to achieve the role defined for the pump connected to the inlet 2; and the capability Interface Function 4 enabling the mixer to control and monitor other optional pumping systems.

All three capabilities are matching with the capability exhibited by the pump.

The negotiation is successful, allowing to start the pairing procedure.

One restriction/condition is defined for the capability exhibited by the pump: “only one system can be paired with the pump to consume this capability”. As no system has yet been paired with the pump to consume this capability, this condition is verified and the pairing can occur.

One restriction/condition is defined for two of the three capabilities exhibited by the mixer: “the confirmation by the operator is required during the pairing”. The pairing will then only be achieved once confirmed by the operator.

A warning message is then displayed by the P&ID GUI of the mixer (not illustrated, for instance on a pop-up window), requesting the operator to assign the pump to one of the three possible usage.

Once the operator has assigned the pump to the inlet 1, the pairing procedure continues.

Both the mixer and the pump memorize the identification and location - OPC UA endpoint - of the paired system for this capability. In the drawn example, only the mixer will use this information to later control and monitor the pump. Once paired, this also avoids the pump from pairing with another system with this capability, too.

On both the mixer and the pump, the DNP manager 19 or 25 publishes the capability description in the (“Control”, “Pumping”) capability data queue. To conform to the selection done by the operator, on the mixer side, this capability is published with the “control application” value set to “Pump_on_inlet1”.

As described above, on both the mixer and the pump, the GUI manager 17 or 23 has subscribed to that capability data queue and automatically updates.

In a non-illustrated variant, instead of removing from the GUI of the pump the Start/Stop button 34 and the variator 35 upon pairing of the pump with an appropriate other system device such as the mixer, only the variator 35 is removed from the GUI of the pump, the Start/Stop button 34 being kept.

### Removal From the Mixer of the Paired Pump and Flow Sensor While the pH Sensor Remains Paired with the Mixer

As mentioned above, the pairing removal of a system device from another system device to which it is paired requires a voluntary and explicit action of the user, so as to enable to distinguish between an intentional disconnection of a system device and a communication failure.

This is carried out here by providing a dedicated menu (not illustrated on the drawings) accessible to the user on the GUI of each system device, so that in the present example each of the P&ID GUI of the mixer, the GUI of the pH sensor, the GUI of the pump and the GUI of the flow sensor has such a dedicated menu.

This dedicated menu lists each other system device with which the system device is paired, and from such menu the user can explicitly request to remove the pairing with a system device selected in the list of the menu.

When the user requests to remove the pairing with a selected other system device, steps are carried out (i) for removing the effects of the step of pairing, (ii) for removing the effects, if any, of the step of negotiation and (iii) for temporarily preventing the system device and the selected other system device to carry out a negotiation step.

For removing the effects of the step of pairing, the DNP manager 19 or 25 of the system device locally publishes the status change of each capability consumed from or provided to the selected other system device and sends to the selected other system device through the MtoM communication tool 18 or 24 a request to proceed to pairing removal. The DNP manager 19 or 25 of the selected other system device in turn locally publishes the status change of each capability consumed from or provided to the system device and sends to the system device through the MtoM communication tool 18 or 24 an acknowledgement receipt of the request to proceed to pairing removal.

In the system device and in the selected other system device, the GUI manager 17 or 23 is warned of the status change of each concerned capability and adapts accordingly.

FIG. 8 illustrates the changes in the P&ID GUI of the mixer, in the GUI of the pump and in the GUI of the flow sensor further to the selection by the user, in the dedicated menu of the mixer, of the pump paired with the flow sensor as to be removed from pairing with the mixer.

Within the mixer, the DNP manager 19 publishes in the appropriate queue, namely the (“Control”, “Pumping”) queue, the status change of the corresponding capability, namely Interface Function 2, the GUI manager 17 is triggered and adapts accordingly by disabling the capability Interface Function 2 so that the control panel 44 is removed from the P&ID GUI of the mixer, as shown at the top of FIG. 8.

Still within the mixer, the DNP manager 19 requests the MtoM communication tool 18 to send to the pump a request to proceed to pairing removal.

This request is transmitted through the network 12, received by the MtoM communication tool 24 of the pump and transferred to the DNP manager 23 of the pump which then publishes in the appropriate queue, namely the (“Control”, “Pumping”) queue, the status change of the corresponding capability, namely Interface Function 2, the GUI manager 23 is triggered and adapts accordingly by putting in original form the capability Interface Function 2 so that the variator 35 and the Start/Stop button 34 become present on the GUI of the pump, as shown at the middle of FIG. 8.

As the flow sensor is not concerned by the pairing removal (it is still paired with the pump), no action is taken thereabout and accordingly its GUI remains unchanged, as shown on FIG. 8 at the bottom. This is the same for the pH sensor which is also not concerned by the pairing removal (it is still paired with the mixer).

The DNP manager 25 of the pump sends to the mixer through the MtoM communication tool 24 an acknowledgement receipt of the request to proceed to pairing removal.

As mentioned above, the effects of the step of negotiation between the mixer and the pump (exclusivity) are invalidated; and for temporarily preventing the mixer and the pump to carry out a negotiation step, because the capabilities previously shared are now again available for negotiation, a quarantine as described earlier is implemented.

The other pairing removals (mixer and pH sensor; pump and flow sensor) are carried out similarly.

### Capability Propagation

As mentioned above, there is in the pump a further capability enabling the pump to provide the flow values sensed by the flow sensor as if the flow sensor was part of the pump.

This further capability, named Interface Function 3, is described in the Device Shape file 26 of the pump.

The description of the capability Interface Function 3 includes an identifier (capabilityUniquelD) and also refers to the identifier (refToCapabilityUniquelD) of another capability, namely the relevant capability of the flow sensor paired with the pump, if any.

It should be noted in this respect that for simplifying the above disclosure, it was not mentioned above that the description of each capability includes an identifier (capabilityUniquelD) which is unique to the capability, in the present example a four-digit value.

### Interface Function 3

When available, Interface Function 3 is similar to the above disclosed interface function in the pH sensor: when in modified form Interface Function 3 of the pump keeps the display of the current value measured by the flow sensor and a trend curve representing the variation of the flow in time while the display is modified for having an indication that the pump is paired with an appropriate other system device, this indication being here the removal of the two control icons 34 and 35 from the GUI of the pump done by Interface Function 2 upon pairing with an appropriate other system device, such as the mixer.

The capability Interface Function 3 has the following description in the Device Shape file 26 of the pump: Domain: “Graphics”, Purpose: “Process Value Display”, Role: “Provider”, Restriction/Condition: NotAvailable. List of properties:

| | |
|---|---|
| Property | Attributes |
| capabilityUniquelD | value=XXXX |
| process_value_dim | mandatory=no, type=text, value=″Flow″ |
| process_value | mandatory=no, type=decimal, access=OpcUa, tag=undefined, source= refToCapabilityUniquelD |
| process_value_name | mandatory=no, type=text, access=OpcUa, tag=undefined, source= refToCapabilityUniquelD |
| process_value_unit | mandatory=no, type=text, access=OpcUa, tag=undefined, source= refToCapabilityUniquelD |

In these, the capabiltyUniquelD property is the identifier unique to Interface Function 3 of the pump; and the tag attributes “tag=undefined, source= refToCapabilityUniquelD” are provided to be updated upon pairing with a flow sensor, with the corresponding attributes in the description of the capability of the flow sensor published in the (“Graphics”, “Process Value Display”) queue within the pump. The Restriction/Condition feature is also updated: it becomes the same as in the capability of the flow sensor; such updating meaning that the capability Interface Function 3 is available.

The description of the capability Interface Function 3 of the pump means: the pump, as a capability provider, is able to provide a flow (and only a flow) Process Value Display dataset to paired system devices using the OPC UA standard. This capability will only be negotiable when the pump will have let it available. The dataset includes a process value, its name and its unit.

Generally speaking, a capability such as Interface Function 3, behaving when activated in the same way as a capability in a paired other system device and as if it was from the system device, can be provided in system devices other than a pump for paired system devices other than a flow sensor.

For mere language convenience, the mechanism involving a capability such as Interface Function 3 of the pump can be termed as capability propagation, with reference to the fact that the source capability (such as the capability in the flow sensor) is rendered available with the same substance through a system device such as the pump, able to activate a capability (such as Interface Function 3) replicating the source capability; and a capability such as Interface Function 3 can be termed a capability propagator.

Of course, since such a capability (such as Interface Function 3) replicates the source capability, the provided capability in the bioprocess machine 13 (here the mixer) replicates the provided capability in the concerned machine helper 14 (here the pump).

### Capability Multiple Sharing

In a variant not illustrated, the pump does not include a capability as Interface Function 3: the flow sensor, instead of being paired only with the pump, is paired together with the pump and with the mixer, so that the mixer gets the flow values directly from the flow sensor (and not from the pump which gets the flow values from the flow sensor).

Indeed, in the capability of the flow sensor, there is no restriction as to the number of other system device with which pairing is authorized while the capability Interface Function 1 of the mixer is able to display process value from several paired system devices, such as the flow sensor in addition to the pH sensor.

For mere language convenience, the fact that a capability (such as the capability in the flow sensor) is consumed by more than one other system device can be termed as capability multiple sharing.

### Capability Hierarchization

In another variant not illustrated, the capability Interface Function 3 is not merely replicating the source capability (the capability in the flow sensor) but enables the pump to provide an additional function the pump is not able to provide without the flow sensor, namely flow regulation.

### Interface Function 3

When available, Interface Function 3 is able to provide controlling and monitoring of the pump speed so as to regulate the flow through the pump using the OPC UA standard.

The capability Interface Function 3 has the following description in the Device Shape file of the pump: Domain: “Control”, Purpose: “PV Regulation”, Role: “Provider”, Restriction/Condition: exclusivity, NotAvailable. List of properties:

| | |
|---|---|
| Property | Attributes |
| capabilityUniquelD | value=XXXX |
| StartStop_Regulation | mandatory=yes, type=boolean, access=OpcUa, tag= opc.tcp://PID/4:StartStop |
| process_value_dim | mandatory=yes, type=text, value=″Flow″ |
| SetPoint | mandatory=no, type= decimal, access=OpcUa, tag=opc.tcp://PID/4:SetPoint |
| Proportional | mandatory=no, type= decimal, access=OpcUa, tag=opc.tcp://PID/4:P |
| Integrated | mandatory=no, type= text, access=OpcUa, tag=opc.tcp://PID/4:I |
| Derived | mandatory=no, type= decimal, access=OpcUa, tag=opc.tcp://PID/4:D |
| CycleTime | mandatory=no, type= decimal, access=OpcUa, tag=opc.tcp://PID/4:cycleTime |
| Deadband | mandatory=no, type= decimal, access=OpcUa, tag=opc.tcp://PID/4:DeadB |
| process_value | mandatory=no, type=decimal, access=OpcUa, tag=undefined, source= refToCapabilityUniquelD |
| process_value_name | mandatory=no, type=text, access=OpcUa, tag=undefined, source= refToCapabilityUniquelD |
| process_value_unit | mandatory=no, type=text, access=OpcUa, tag=undefined, source= refToCapabilityUniquelD |
| process_value_min_range | mandatory=no, type= decimal, access=OpcUa, tag=undefined, source= refToCapabilityUniquelD |
| process_value_max_range | mandatory=no, type= decimal, access=OpcUa, tag=undefined, source= refToCapabilityUniquelD |

In these, the capabiltyUniquelD property is the identifier unique to Interface Function 3 of the pump; and the tag attributes “tag=undefined, source= refToCapabilityUniquelD” are provided to be updated upon pairing with a flow sensor, with the corresponding attributes in the description of the capability of the flow sensor published in the (“Graphics”, “Process Value Display”) queue within the pump. The Restriction/Condition feature is also updated for stating that the capability Interface Function 3 is available.

The description of the capability Interface Function 3 of the pump means: the pump, as a capability provider with Process Value Regulation skills is able to provide controlling and monitoring of the pump speed so as to regulate the flow through the pump using the OPC UA standard. This capability will only be negotiable when the pump will have let it available. The paired system device, such as the mixer, will then have the exclusivity of the control and will be able to Start/Stop the regulation, to set the regulation parameters and to retrieve data from the process value.

Generally speaking, a capability such as Interface Function 3, providing when activated an additional function, can be provided in system devices other than a pump for paired system devices other than a flow sensor.

For mere language convenience, the mechanism involving a capability such as Interface Function 3 of the pump can be termed as capability hierachization, with reference to the fact that the source capability (such as the capability in the flow sensor) is embedded in a more complex capability; and a capability such as Interface Function 3 can be termed a hierarchized capability.

Though such a capability (such as Interface Function 3) may include the source capability, the provided capability in the bioprocess machine 13 (here the mixer) need to necessarily embed the provided capability in the concerned machine helper 14 (here the pump).

As used herein, the term “hierarchical capability” is generally used to denote that a source capability is mandatory for a machine helper to be able to expose / show the hierarchical capability. For example, “volume calculation” as hierarchical capability would, for example and depending on the shape of the volume to be determined, require “length”, “height” and “depth” as source capabilities.

### Variant of the System Devices With Recipe Manager

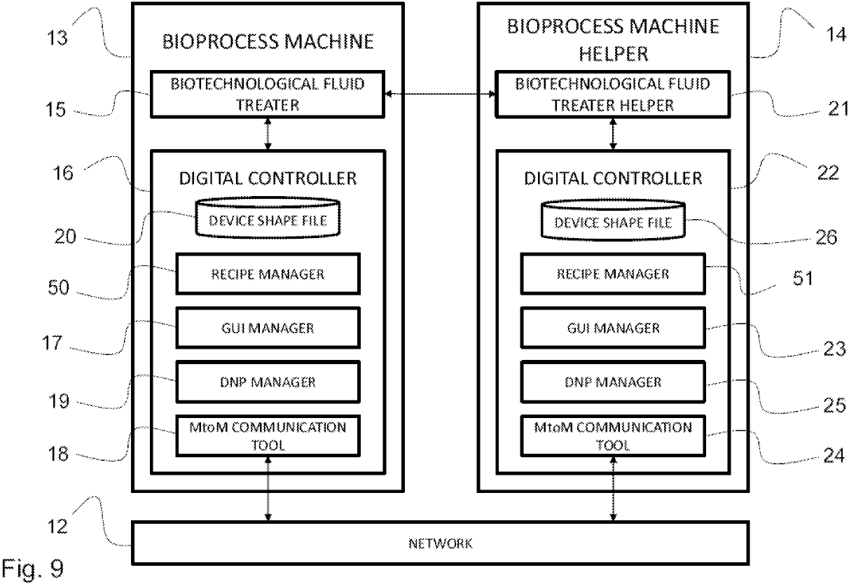

FIG. 9 shows in the same way as FIG. 3 a variant of the system devices in which the digital controller 16 of the bioprocess machine 13 further includes a recipe manager 50 and the digital controller 22 of the machine helper 14 further includes a recipe manager 51.

In the bioprocess machine 13, the recipe manager 50 allows to display a GUI (as shown on FIGS. 11 to 13) locally on an interactive screen or remotely on a device having an interactive screen, for e.g., a tablet or smartphone.

In the machine helper 14, the recipe manager 51 is not configured for displaying a GUI.

### Recipe Management

Important notions in automation and more particularly in recipes are criteria (testable values) and actions.

A recipe mainly consists in an ordered set of instructions that will cause actions on the controlled device (like starting motors, setting heating set point, changing alarms conditions, setting controller constants...) or acquisitions of data from the controlled device to be evaluated as criteria for test (like process value, machine state, alarm state...).

A recipe can include recipe execution control that will define in which order the instructions have to be executed (e.g. loop instruction, conditional branching ...).

The following is an example of recipe:

“While (pH > 6) do [= recipe execution control and criteria evaluation]
Inlet1.Valve.Open TRUE [= action]
Wait 500 ms [= recipe execution control]
Inlet1.Valve.Open FALSE [= action]
If (pH < 3) do [= recipe execution control and criteria evaluation]
Prompt ‘Too acid’ [= action]
Else [= recipe execution control and criteria evaluation]
Prompt ‘pH OK’ [= action]”

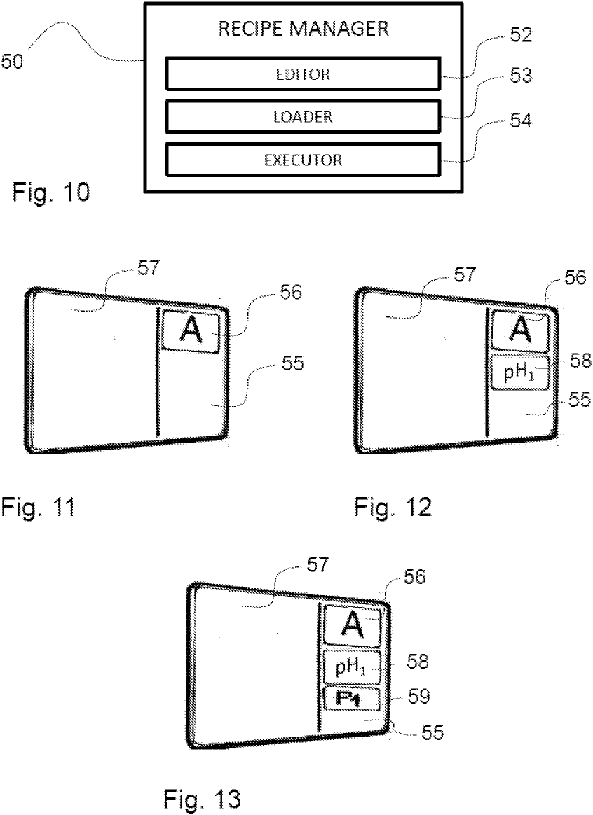

As shown on FIG. 10, the recipe manager 50 of the bioprocess machine 13 has three modules, respectively an editor 52, a loader 53 and an executor 54.

The editor 52 allows a user to create recipes, a human readable way, assembling the available instructions for the bioprocess machine 13.

A recipe can be selected to be ran on the bioprocess machine 13.

The loader 53 translates the human readable instructions set into a machine-oriented instructions list and then loads the instructions list into the executor 54 for execution of the translated instructions in the predefined order.

Generally speaking, the recipe manager 50 of the bioprocess machine 13 and the recipe manager 51 of the machine helper 14 are configured such that in paired condition:
the recipe manager 50 of the bioprocess machine 13 has at least one provided capability that it does not have when not in paired condition, said provided capability being an automation function controlling and/or testing an operating parameter of the treater helper 21 or testing a physico-chemical or biological quantity of the fluid sensed by the treater helper 21; and
the recipe manager 51 of the machine helper 14 has at least one consumed capability that it does not have when not in paired condition, wherein: if said provided capability is said automation function controlling and/or testing an operating parameter of the treater helper 21 said consumed capability is a response-on-request function controlling and/or making available said operating parameter, and if said provided capability is said automation function testing a physico-chemical or biological quantity of said fluid sensed by the treater helper 21 said consumed capability is a response-on-request function making available said physico-chemical or biological quantity.

Each such automation function is a recipe instruction, such controlling of an operating parameter is an action, and such operating parameter, physico-chemical quantity or biological quantity which can be tested is a criteria.

In practice, the automation function or response-on-request function is present but disabled (or not used) when the system device 13 or 14 is not paired with an appropriate other system device and enabled (or put into use) when the system device 13 or 14 is paired with an appropriate other system device.

A description will now be given of the operation of the recipe manager 50 when the mixer is in stand-alone condition (FIG. 11), and of the operation of the recipe managers 50 and 51 when the mixer is paired with the pH sensor (FIG. 12) and when the mixer is paired with the pH sensor and further paired with the pump previously paired with the flow sensor (FIG. 13).

### Operation of the Recipe Manager 50 When the Mixer Is in Stand-alone Condition

As mentioned above, the recipe manager 50 allows to display a GUI locally on an interactive screen or remotely on a device having an interactive screen, for e.g., a tablet or smartphone.

FIG. 11 shows this GUI when the mixer is in stand-alone condition.

On this GUI there is a section 55 in which there is a list 56 of actions, properties and/or criteria corresponding to the mixer.

When the mixer is in stand-alone condition, there is only the list 56 in the section 55.

The GUI of the recipe manager 50 also includes in the section 57 the GUI of editor 52.

On this window, there is a set of instructions related to the control and the monitoring of the biotechnological fluid treater 15 of the mixer, formed as disclosed above by a tank with an agitator and inlets.

This set of instructions is based on the actions and criteria that the mixer has natively, such as but not limited to the actions “start agitator”, “set agitator speed”, “set all alarms off” and the criteria “agitator speed”.

These actions and criteria are described in the Device Shape file 20 of the mixer.

With this window, the user can create recipes using the available instructions, and can store the created recipes.

On the GUI of the recipe manager 50, there is a menu (not illustrated) allowing to select one of the stored recipes to be loaded for execution.

During this operation, because a recipe can be loaded at any moment, the loader 53 checks if the instructions used in the recipe are still defined for the mixer, then it translates the operator-entered instructions into machine-executable instructions and loads the result into the executor 54.

From the GUI of the recipe manager 50, the user can start the execution of the recipe. The executor 54 will then automatically control and monitor the mixer, sequentially executing the translated instructions in the order specified by the recipe.

### Operation of the Recipe Managers 50 and 51 When the Mixer Is Paired with the pH Sensor

FIG. 12 shows the GUI of the recipe manager 50 when the mixer is paired with the pH sensor.

In the section 55, further to the icon 56 corresponding to the mixer there is a list 58 of actions, properties and/or criteria corresponding to the pH sensor.

On the window (not illustrated) which is the GUI of the editor 52, further to the set of instructions related to the control and the monitoring of the biotechnological fluid treater 15 of the mixer, there is at least one instruction allowing to monitor (by testing) the pH value provided by the pH sensor and more precisely sensed by the treater helper 21 of the pH sensor.

The recipe manager 51 of the pH sensor is involved in the mechanism enabling to monitor the pH value provided by the pH sensor, the recipe manager 51 providing on request a response making available the current pH value. This will be explained in detail later.

The operation of the recipe manager 50 is the same as when the mixer is in stand-alone condition, except the availability of the further instruction(s) allowing to monitor the pH value provided by the pH sensor.

As mentioned above, the user can create recipes using the available instructions, and can store the created recipes.

On the GUI of the recipe manager 50, there is a menu (not illustrated) allowing to select one of the stored recipes to be loaded for execution.

During this operation, because a recipe can be loaded at any moment, the loader module 53 checks if the instructions used in the recipe are still defined for the mixer, in particular if a pH sensor is still paired with the mixer if there is an instruction monitoring pH, then it translates the instructions and loads the result into the executor 54.

From the GUI of the recipe manager 50, the user can start the execution of the recipe. The executor 54 will then automatically control and monitor the mixer and pH sensor, sequentially executing the translated instructions in the order specified by the recipe.

### Operation of the Recipe Managers 50 and 51 When the Mixer Is Paired with the pH Sensor and Further Paired with the Pump Previously Paired with the Flow Sensor

FIG. 13 shows the GUI of the recipe manager 50 when the mixer is paired with the pH sensor and further paired with the pump previously paired with the flow sensor.

In the section 55, further to the list 56 corresponding to the mixer and the list 58 corresponding to the pH sensor there is a further list 59 of actions, properties and/or criteria corresponding to the pump.

On the window (not illustrated) which is the GUI of the editor 52, further to the set of instructions related to the control and the monitoring of the biotechnological fluid treater 15 of the mixer, there are further instructions allowing to monitor the pH value provided by the pH sensor and more precisely sensed by the treater helper 21 of the pH sensor, to monitor (by testing) the flow value provided by the flow sensor and more precisely sensed by the treater helper 21 of the flow sensor, and to control and monitor (by testing) the pump and more precisely the treater helper 21 of the pump.

The recipe managers 51 of the pH sensor, flow sensor and pump are involved in the mechanism enabling to monitor the pH value, the flow value and to control and monitor the operating parameters of the pump, each recipe manager 51 providing on request a response making available the current pH value, flow value or other criteria; or providing on request a response controlling and/or making available an operating parameter of the pump. This will be explained in detail later.

The operation of the recipe manager 50 is the same as when the mixer is in stand-alone condition, except the availability of the further instructions allowing to monitor the pH value provided by the pH sensor, to monitor the flow value provided by the flow sensor and to control and monitor the pump.

As mentioned above, the user can create recipes using the available instructions, and can store the created recipes.

On the GUI of the recipe manager 50, there is a menu (not illustrated) allowing to select one of the stored recipes to be loaded for execution.

During this operation, because a recipe can be loaded at any moment, the loader 53 checks if the instructions used in the recipe are still defined for the mixer, in particular if the needed pH sensor and pump are still paired with the mixer, then it translates the instructions and loads the result into the executor 54.

From the GUI of the recipe manager 50, the user can start the execution of the recipe. The executor 54 will then automatically control and monitor the mixer, pH sensor, pump and flow sensor sequentially executing the translated instructions in the order specified by the recipe.

### Recipe Domain

In the above description of the system devices 13 or 14 without recipe manager 50 or 51, given with regard to FIGS. 3 to 8, all capabilities are interface functions, respectively of the GUI manager 17 of the bioprocess machine 13 and of GUI manager 23 of the machine helper 14, the Domain feature being “Graphics” or “Control”.

All that has been said above about capabilities for the GUI managers 17 and 23 applies to the recipe managers 50 and 51 mutatis mutandis, in particular instead of being an interface function a capability is an automation function for the recipe manager 50 of the bioprocess machine 13 and a response-on-request function for the recipe manager 51 of the bioprocess machine 14, the Domain feature being “Recipe”.

In the example illustrated on the drawings, for the bioprocess machine 13 which is a mixer, the Device Shape file 20 further contains a description of two automation functions which are provided capabilities when enabled; and for the machine helpers 14 which are respectively a pH sensor, a pump and a flow sensor the Device Shape file 26 contains a description of at least one response-on-request function which is a consumed capability when enabled.

### Automation Function 1 of the Mixer

When enabled, Automation Function 1 of the mixer allows to extend the recipe management capability with new criteria provided by paired system devices, whatever the paired system devices are. When used in a recipe, Automation Function 1 allows the bioprocess machine which is a here a mixer to automatically retrieving data from the machine helper 14.

The capability Automation Function 1 has the following description in the Device Shape file 20 of the mixer: Domain: “Recipe”, Purpose: “Criteria”, Role: “Consumer”, Restriction/Condition: optional. List of properties:

| | |
|---|---|
| Property | Attributes |
| shortDescription | mandatory=yes, type=text, access=unconstrained |
| valueUnit | mandatory=yes, type=text, access=unconstrained |
| valueMinRange | mandatory=yes, type=decimal, access=unconstrained |
| valueMaxRange | mandatory=yes, type=decimal, access=unconstrained |
| translatedInstruction | mandatory=yes, type=text, access=unconstrained |
| requestAddress | mandatory=yes, type=text, access=opcUa |
| responseAddress | mandatory=yes, type=text, access=opcUa |
| capabilityUniquelD | value=XXXX |

This capability description means: the mixer, as a capability consumer with recipe skills, is able to retrieve process values from paired system devices, and to use them as test criteria in a recipe. The paired system devices will mandatorily have to provide the data for the criteria: the information to be used in the editor 52 (from property shortDescription to property valueMaxRange) and the information to be used by the loader 53 and the executor 54 to execute the recipe (translatedInstruction, requestAddress, responseAddress). Some data have to be provided as OPC UA tags. Some data (unconstrained) can be provided as needed by the capability provider. No restriction or condition are imposed for the negotiation or pairing.

It should be noted that many more properties than mentioned above can be included in the information to be used by the editor 52 and in the information to be used by the loader 53 and the executor 54.

It should further be noted that instead of providing a capability for each criteria, it is possible to provide a capability for a set of criteria. Indeed, as a machine helper 14 can potentially propose tens of recipe criteria, it is more efficient for negotiation to provide a single capability that will provide the data for the recipe criteria all at once.

### Automation Function 2 of the Mixer

Generally speaking, Automation Function 2 is similar to Automation Function 1 except that it is for actions instead of criteria.

When enabled, Automation Function 2 of the mixer allows to extend the recipe management capability with new actions provided by paired system devices, whatever the paired system devices are. When used in a recipe, Automation Function 2 allows to automatically control the machine helper 14 from the bioprocess machine 13 which is here a mixer.

The capability Automation Function 2 has the following description in the Device Shape file 20 of the mixer: Domain: “Recipe”, Purpose: “Action”, Role: “Consumer”, Restriction/Condition: optional. List of properties:

| | |
|---|---|
| Property | Attributes |
| shortDescription | mandatory=yes, type=text, access=unconstrained |
| argumentUnit | mandatory=yes, type=text, access=unconstrained |
| argumentMinRange | mandatory=yes, type=decimal, access=unconstrained |
| argumentMaxRange | mandatory=yes, type=decimal, access=unconstrained |
| translatedInstruction | mandatory=yes, type=text, access=unconstrained |
| requestAddress | mandatory=yes, type=text, access=opcUa |
| responseAddress | mandatory=yes, type=text, access=opcUa |
| capabilityUniquelD | value=XXXX |

This capability description means: the mixer, as a capability consumer with recipe skills, is able to cause actions on paired system devices, and to use them in a recipe. The paired system devices will mandatorily have to provide the data for the action: the data to be used in the editor 52 (from property shortDescription to property argumentMaxRange) and the data to be used by the loader 53 and the executor 54 to execute the recipe (translatedlnstruction, requestAddress, responseAddress). Some data have to be provided as OPC UA tags. Some data (unconstrained) can be provided as needed by the capability provider. No restriction or condition are imposed for the negotiation or pairing.

It should be noted that many more properties than mentioned above can be included in the information to be used by the editor 52 and in the information to be used by the loader 53 and the executor 54.

It should further be noted that instead of providing a capability for each action, it is possible to provide a capability for a set of actions. Indeed, as a machine helper can potentially propose tens of recipe actions, it is more efficient for negotiation to provide a single capability that will provide the data for the recipe actions all at once.

### Response-on-request Function of the pH Sensor

When enabled, Response-on-request Function of the pH sensor allows to provide new criteria to the paired system device so as to extend its recipe management capability, whatever the paired system device is, though primarily this is the process machine.

The capability Response-on-request Function of the pH sensor has the following description in the Device Shape file 26 of the pH sensor: Domain: “Recipe”, Purpose: “Criteria”, Role: “Provider”, Restriction/Condition: none. List of properties:

| | |
|---|---|
| Property | Attributes |
| shortDescription | mandatory=yes, type=text, access=asValue, value=″pH″ |
| valueUnit | mandatory=no, type=text, access=OpcUa, tag=opc.tcp://ph/4:control/4:unit |
| valueMinRange | mandatory=no, type=decimal, access=OpcUa, tag=opc.tcp://ph/4:control/4:minRange |
| valueMaxRange | mandatory=no, type=decimal, access=OpcUa, tag=opc.tcp://ph/4:control/4:maxRange |
| translatedInstruction | mandatory=yes, type=text, access=asValue, value=″1~11~1″ |
| requestAddress | mandatory=yes, type=text, access=opcUa, tag=opc.tcp://ph/4:control/4:request |
| responseAddress | mandatory=yes, type=text, access=opcUa, tag=opc.tcp://ph/4:control/4:answer |
| capabilityUniquelD | value=XXXX |

This capability description means: the pH sensor, as a capability provider with recipe skills, proposes its pH value to be used as a criteria in a recipe executed on the executor 54 of the consumer. The consumer has mandatorily to request and/or wait for (and then understand) the translatedInstruction, requestAddress, responseAddress properties from the provider, to be able to access the pH value. No restriction or condition are imposed for the negotiation or pairing.

It should be noted that many more properties than mentioned above can be included in the information to be used by the editor 52 (from property shortDescription to property valueMaxRange) and in the information to be used by the loader 53 and the executor 54 (translatedInstruction, requestAddress, responseAddress).

It should further be noted that instead of providing a capability for each criteria, it is possible to provide a capability for a set of criteria. Indeed, as a machine helper can potentially propose tens of recipe criteria, it is more efficient for negotiation to provide a single capability that will provide the data for the recipe criteria all at once.

### Response-on-request Function of the Flow Sensor

When enabled, Response-on-request Function of the flow sensor allows to provide new criteria to the paired system device so as to extend its recipe management capability, whatever the paired system device is.

The capability Response-on-request Function of the flow sensor has the following description in the Device Shape file 26 of the flow sensor: Domain: “Recipe”, Purpose: “Criteria”, Role: “Provider”, Restriction/Condition: none. List of properties:

| | |
|---|---|
| Property | Attributes |
| shortDescription | mandatory=yes, type=text, access=asValue, value=″Flow″ |
| valueUnit | mandatory=no, type=text, access=OpcUa, tag=opc.tcp://flow/4:control/4:unit |
| valueMinRange | mandatory=no, type=decimal, access=OpcUa, tag=opc.tcp://flow/4:control/4:minRange |
| valueMaxRange | mandatory=no, type=decimal, access=OpcUa, tag=opc.tcp://flow/4:control/4:maxRange |
| translatedInstruction | mandatory=yes, type=text, access=asValue, value=“1~12~1″ |
| requestAddress | mandatory=yes, type=text, access=opcUa, tag=opc.tcp://flow/4:control/4:request |
| responseAddress | mandatory=yes, type=text, access=opcUa, tag=opc.tcp://flow/4:control/4:answer |
| capabilityUniquelD | value=XXXX |

This capability description means: the flow sensor, as a capability provider with recipe skills, proposes its flow value to be used as a criteria in a recipe executed on the executor 54 of the consumer. The consumer has mandatorily to request and/or wait for (and then understand) the translatedInstruction, requestAddress, responseAddress properties from the provider, to be able to access the flow value. No restriction or condition are imposed for the negotiation or pairing.

It should be noted that many more properties than mentioned above can be included in the information to be used by the editor 52 (from property shortDescription to property valueMaxRange) and in the information to be used by the loader 53 and the executor 54 (translatedInstruction, requestAddress, responseAddress).

It should further be noted that instead of providing a capability for each criteria, it is possible to provide a capability for a set of criteria. Indeed, as a machine helper can potentially propose tens of recipe criteria, it is more efficient for negotiation to provide a single capability that will provide the data for the recipe criteria all at once.

### Response-on-request Function 1 of the Pump

Generally speaking, the capability Response-on-request Function 1 of the pump is similar to the capability response-on-request Function of the pH sensor or of the flow sensor, except that the machine helper 14 is the pump (and not the pH sensor or the flow sensor) and that the criteria is the motor speed of the pump (and not the pH value or the flow value).

### Response-on-request Function 2 of the Pump

When enabled, Response-on-request Function 2 of the pump allows to provide new action(s) to the paired system device so as to extend its recipe management capability, whatever the paired system devices are.

The capability Response-on-request Function 2 of the pump has the following description in the Device Shape file 26 of the pump: Domain: “Recipe”, Purpose: “Action”, Role: “Provider”, Restriction/Condition: none. List of properties:

| | |
|---|---|
| Property | Attributes |
| shortDescription | mandatory=yes, type=text, access=asValue, value=″Pump->Start″ |
| argumentUnit | mandatory=yes, type=text, access=asValue, value=⁗ |
| argumentMinRange | mandatory=yes, type=decimal, access=asValue, value=″ |
| argumentMaxRange | mandatory=yes, type=decimal, access=asValue, value=⁗ |
| translatedInstruction | mandatory=yes, type=text, value=″5~1~1″ |
| requestAddress | mandatory=yes, type=text, access=opcUa, tag=opc.tcp://Pump/4:control/4:request |
| responseAddress | mandatory=yes, type=text, access=opcUa, tag=opc.tcp://Pump/4:control/4:answer |
| capabilityUniquelD | value=XXXX |

This capability description means: the pump, as a capability provider with recipe skills, proposes its pump to be started from a recipe executed on the executor 54 of the consumer. The consumer has mandatorily to request and/or wait for (and then understand) the translatedInstruction, requestAddress, responseAddress properties from the provider, to be able to start/stop the pump motor, as is possible with the Start/Stop button 34. No restriction or condition are imposed for the negotiation or pairing.

It should be noted that many more properties than mentioned above can be included in the information to be used by the editor 52 (from property shortDescription to property valueMaxRange) and in the information to be used by the loader 53 and the executor 54 (translatedInstruction, requestAddress, responseAddress).

### Response-on-request Function 3 of the Pump

Generally speaking, the capability Response-on-request Function 3 of the pump is similar to the capability Response-on-request Function 2, except that the action is to set the motor speed, as is possible with the variator 35.

### Response-on-request Function 4 of the Pump

Generally speaking, the capability Response-on-request Function 4 of the pump is similar to the capability response-on-request Function 2, except that the action is the flow regulation, as is possible with the capability Interface Function 3 of the pump in its version as hierarchized capability. Response-on-request Function 4 is also a hierarchized capability.

It should be noted be that instead of providing a capability for each action, it is possible to provide a capability for a set of actions. Indeed, as a machine helper can potentially propose tens of recipe actions, it is more efficient for negotiation to provide a single capability that will provide the data for the recipe actions all at once.

It should be further noted that in a variant, Response-on-request Function 4 of the pump is similar to the capability response-on-request Function 1, except that the criteria is the flow value provided by the flow sensor, as is possible with the capability Interface Function 3 of the pump in its version as capability propagator. In this variant, response-on-request Function 4 is also a capability propagator.

In a further variant, the pump has no capability as Request-on-response Function 4 and the flow sensor is paired together with the pump and with the mixer, its response-on-request function being a multiple shared capability.

### DNP Sequence

During the different pairings, the DNP sequence is carried out by the recipe managers 50 and 51 as described above for the GUI managers 17 and 23, the Domain feature involved being of course at “Recipe”.

Just as the GUI managers 17 and 23, the recipe manager 50 of the mixer subscribes to the data queues with a Domain feature at “Recipe”.

When the negotiation/pairing with the pH sensor has succeeded, the DNP manager 19 of the mixer publishes the description of the pH sensor criteria capability in the (“Recipe”, “Criteria”) queue. The recipe manager 50 is warned a new capability has been published in this queue and it can then extend its instructions set using the description provided in this queue.

When the negotiation/pairing with the pump has succeeded, the DNP manager 19 of the mixer publishes the description of the pump criteria capability in the (“Recipe”, “Criteria”) queue and the description of the pump action capability in the (“Recipe”, “Action”) queue. The recipe manager 50 is warned new capabilities have been published in these queues and it can then extend its instructions set using the description provided with these queues.

In the treater helpers 14 (pH sensor, flow sensor and pump), the recipe manager 51 does not subscribe to the data queues with a Domain feature at “Recipe”, because pairing is sufficient for transitioning the response-on-request function of the recipe manager 51 from disabled (no other system device is able to make a request thereto) to enabled (upon pairing the paired system device becomes able to make a request thereto).

### Loading and Execution of a Recipe

As mentioned above, on the GUI of the recipe manager 50, there is a menu (not illustrated) allowing to select one of the stored recipes to be loaded for execution.

During this operation, because a recipe can be loaded at any moment, the loader 53 optionally checks if the instructions used in the recipe are still defined for the mixer: if the recipe includes at least one instruction involving a given paired system device, the loader 53 checks if this system device is still paired with the mixer.

If this checking is positive, the loader 53 creates a communication path between MtoM Communication Tool 18 of the bioprocess machine 13, which is here a mixer, and MtoM Communication Tool 24 of the machine helper 14, which is here a pH sensor, a flow sensor or a pump.

The communication path allows the executor 54 to access to the criteria value in the paired machine helper 14 or to send a request for action to the paired machine helper 14.

This is done thanks to the requestAddress and responseAddress properties in the concerned automation function of the mixer, updated upon pairing with the appropriate machine helper 14, for instance for the pH sensor tag=opc.tcp://ph/4:control/4:request as requestAddress property and tag=opc.tcp://ph/4:control/4:answer as responseAddress property.

The loader 53 translates each instruction of the recipe into a machine-oriented instruction and loads the translation into the executor 54.

An instruction controlling/monitoring an instrument of the fluid treater 15 of the bioprocess machine 13 such as “agitator.start” is so translated: if the recipe instruction involves an action, it is translated into an instruction writing a value directly to the instrument of the fluid treater 15; and if the recipe instruction involves a criteria, it is translated into an instruction reading a value directly from the instrument of the fluid treater 15.

An instruction controlling/monitoring an instrument of the treater helper 21 of a paired machine helper 14, such as “pump.start” or “flow sensor.flow”, is so translated: if it is a recipe instruction involving an action, it is translated into an instruction sending the translatedInstruction property value through the request channel of the communication path; and if it is a recipe instruction involving a criteria, into an instruction sending the translatedInstruction property value through the request channel of the communication path and an instruction reading the value through the response channel of the communication path.

The translation is done thanks to the translatedInstruction property in the concerned automation function of the mixer, updated upon pairing with the appropriate machine helper 14.

A recipe instruction involving a criteria such as “While (pH > 6) do” is translated into “Submit pH request on tag Y” and “While (tag X < 6) do” with tag Y which is the requestAddress property and tag X which is the responseAddress property.

As mentioned above, upon publication of the description of the pH sensor criteria capability in the (“Recipe”, “Criteria”) queue created by the DNP manager 19 of the mixer, the recipe manager 50 of the mixer is automatically notified and receives the description of the pH sensor criteria capability, including the OPC UA tags in the requestAddress property and in the responseAddress property, so that the recipe manager 50 is able with the loader 53 to create a communication path between the mixer and the pH sensor, thanks to the OPC UA client in the MtoM communication tool 18 of the mixer, the network 12 and the OPC UA server in the MtoM communication tool 24 of the pH sensor.

The instruction “Submit pH request on tag Y” is carried out by the executor 54 by asking the MtoM communication 18 to send with its OPC UA client the pH request through the network 12 to the OPC UA tag in the requestAddress property (namely opc.tcp://ph/4:control/4:request).

The instruction “While (tag X < 6) do” is carried out by the executor 54 via the communication path between the MtoM Communication Tool 18 and the MtoM Communication Tool 24 to retrieve with its OPC UA client the pH value through the network 12 at the OPC UA tag in the responseAddress property (namely opc.tcp://ph/4:control/4:answer).

Turning now to a recipe instruction involving an action such as “Pump->Start”, such instruction is translated into “Set tag Y with value Z” in which tag Y is the requestAdress property and value Z is the translatedInstruction property.

As mentioned above, upon publication of the description of Response-on-Request Function 2 capability of the pump in the (“Recipe”, “Criteria”) queue created by the DNP manager 19 of the mixer, the recipe manager 50 of the mixer is automatically notified and receives the description of Response-on-Request Function 2 capability of the pump, including the translatedInstruction property and the OPC UA tag in the requestAddress property.

The recipe manager 50 is able with the loader 53 to create a communication path between the mixer and the pump, thanks to the OPC UA client in the MtoM communication tool 18 of the mixer, the network 12 and the OPC UA server in the MtoM communication tool 24 of the pump.

The instruction “Set tag Y with value Z” is carried out by the executor 54 via the communication path between the MtoM Communication Tool 18 and the MtoM Communication Tool 24 to send with its OPC UA client the value Z (namely “5~1~1”) through the network 12 to the OPC UA tag in the requestAddress property (namely opc.tcp://Pump/4:control/4:request).

From the GUI of the recipe manager 50, the user can start the execution of the recipe. The executor 54 will then automatically control and monitor the appropriate machine helper 14, such as a pH sensor, pump or flow sensor, sequentially executing the translated instructions in the order specified by the recipe.

As mentioned above, a criteria instruction such as “Submit pH request on tag Y” is carried out by the executor 54 by asking the MtoM communication 18 to send with its OPC UA client the pH request through the network 12 to the OPC UA tag in the requestAddress property (namely opc.tcp://ph/4:control/4:request).

When the OPC UA server in the MtoM communication tool 24 of the pH sensor receives this request, the MtoM communication tool 24 informs accordingly the recipe executor 54 of the recipe manager 51 of the pH sensor. In reaction to this request, thanks its response-on-request function, the recipe executor 54 of the recipe manager 51 of the pH sensor, which is provided in real time with the pH value sensed by the treater helper 21 of the pH sensor, makes the pH value available on the network 12 thanks to the OPC UA server of the MtoM communication tool 24 of the pH sensor at the OPC UA tag given in the in the responseAddress property (namely opc.tcp://ph/4:control/4:answer).

Accordingly, the recipe executor 54 of the recipe manager 51 can carry out the instruction “While (tag X < 6) do” via the communication path between the MtoM Communication Tool 18 and the MtoM Communication Tool 24 to retrieve with its OPC UA client the pH value through the network 12 at the OPC UA tag in the responseAddress property (namely opc.tcp://ph/4:control/4:answer).

As mentioned above, an action instruction such as “Set tag Y with value Z” is carried out by the recipe executor 54 of the recipe manager 51 via the communication path between the MtoM Communication Tool 18 and the MtoM Communication Tool 24 to send with its OPC UA client the value Z (namely “5~1~1”) through the network 12 to the OPC UA tag in the requestAddress property (namely opc.tcp://Pump/4:control/4:request).

When the OPC UA server in the MtoM communication tool 24 of the pump receives this request, the MtoM communication tool 24 informs accordingly the recipe executor 54 of the recipe manager 51 of the pump. In reaction to this request, thanks its Response-on-request Function 2 capability, the recipe manager 51 of the pump writes the value Z (namely “5~1~1”) in the treater helper 21 of the pump, so that the pump motor starts.

In a variant, instead of merely writing the value Z in the treater helper 21, the recipe manager 51 sends a feed-back message to the recipe manager 50 that the pump is instructed to start, by making the feed-back message available on the network 12 thanks to the OPC UA server of the MtoM communication tool 24 of the pump at the OPC UA tag given in the in the responseAddress property (namely opc.tcp://Pump/4:control/4:answer).

Of course, the above described mechanism for a criteria instruction of the pH sensor applies to other machine helpers 14 providing a criteria, for instance to the pump providing the motor speed as criteria; and the above described mechanism for an action instruction of the pump applies to other machine helpers 14 providing an action, for instance a valve providing closing or opening as action.

Pairing removal is carried out as described above.

In a non-illustrated variant, there is no GUI manager 17 or 23 in the system devices 13 or 14, the user interfaces being carried out differently.

Further variants

In variants to the examples disclosed above:
the bioprocess machine is different from a mixer, for instance a bioreactor, a chromatograph, a virus inactivation or a tangential flow filtration;
the machine helpers are different from a pump, a flow sensor and a pH sensor, for example other active components such as valves or mass flow controllers; other sensors whether mechanical, electronic, opto-electronic, infrared, ultra-violet, etc. like pressure sensors, temperature sensors, OD (Optical Density) sensors, DO (Dissolved Oxygen) sensors, gas (CO2, ...) sensors, weight sensors, speed (RPM...) sensors, flow (gas/air) rate sensors, humidity sensors, hygrometry sensors, light/lux sensors, position (valve, actuator, switch...) sensors, power (watt..) sensors, galvanometers, motion sensors, vacuum sensors, title sensors, viability sensors, resistivity sensors, proximity/distance sensors, volume sensors, UV sensors, IR sensors, frequency sensors, molar concentration sensors, duration/time sensors, radiation sensors, colorimeters, glucometers, opacimeters, osmometers, photometers, spectroscopes, acoustic pressure sensors, sonometers, video sensors, photo sensors, electrical charge sensors, particle counters, viscosity sensors or lactate sensors; other types of instrumentation; and/or ancillary devices such as a cell retention device or a mixer which are capability providers (unlike the mixer in the above example);
unlike the mixer in the above example for which it is mandatory to be paired with a pump connected to inlet 1 for being operable, the bioprocess machine is operable in stand-alone condition, that is to say without having to be paired to a bioprocess machine helper for being operable;
the system devices are originally in places different from a production area and a storage area, for instance all system devices are originally in a storage area and they are all brought in a production area for setting up the installation;
the interactive screen is replaced or complemented by another user interface such as a passive display and physical buttons or a passive screen and a keyboard;
the network is different from a network with IP;
the machine to machine communication standard is different from OPC UA; and/or
in the system there is only one bioprocess machine and one bioprocess machine helper; or there is a plurality of bioprocess machines and a plurality of bioprocess machine helpers with at least certain machine helpers which can be paired with different bioprocess machines.

Many other variants are possible and it is recalled in this respect that the invention is not limited to the examples disclosed and illustrated.

1. System for treating a biotechnological fluid, having the following system devices:
a bioprocess machine (13) having: a biotechnological fluid treater (15) configured for modifying at least one physico-chemical or biological property of said biotechnological fluid and a digital controller (16) for controlling said biotechnological fluid treater (15); and
at least one bioprocess machine helper (14) having: a biotechnological fluid treater helper (21) configured for being physically coupled to said biotechnological fluid treater (15) and a digital controller (22) for controlling said biotechnological fluid treater helper (21); wherein:
said digital controller (16) of said bioprocess machine (13) and said digital controller (22) of said machine helper (14) each include a recipe manager (50, 51), a machine to machine communication tool (18, 24) (MtoM communication tool), and a discovery negotiation pairing manager (19, 25) (DNP manager);
each said MtoM communication tool (18, 24) is configured for connecting to a network (12);
the DNP manager (19) of said bioprocess machine (13) and the DNP manager (25) of said machine helper (14) are configured for cooperating over said network (12) for establishing a paired condition; wherein the recipe manager (50) of the bioprocess machine (13) and the recipe manager (51) of the machine helper (14) are configured such that in said paired condition:
the recipe manager (50) of the bioprocess machine (13) has at least one provided capability that it does not have when not in paired condition, said provided capability being an automation function controlling and/or testing an operating parameter of said treater helper (21) or testing a physico-chemical or biological quantity of said fluid sensed by said treater helper (21); and
the recipe manager (51) of the machine helper (14) has at least one consumed capability that it does not have when not in paired condition, wherein: if said provided capability is said automation function controlling and/or testing an operating parameter of the treater helper (21) said consumed capability is a response-on-request function controlling and/or making available said operating parameter, and if said provided capability is said automation function testing a physico-chemical or biological quantity of said fluid sensed by the treater helper (21) said consumed capability is a response-on-request function making available said physico-chemical or biological quantity.

2. System according to claim 1, wherein the recipe manager (50) of the bioprocess machine (13) is configured for displaying a graphical user interface, said graphical user interface of the recipe manager (50) of the bioprocess machine (13) being configured for allowing a user to create recipes with said provided capability automation function, to store so created recipes, to load so stored recipes and to execute so loaded recipes.

3. System according to claim 1, wherein said recipe manager (51) of the machine helper (14) is not configured for displaying a graphical user interface.

4. System according to claim 1, wherein said provided capability automation function is controlling and/or testing a set of operating parameters of said treater helper (21) or testing a set of physico-chemical or biological quantity of said fluid sensed by said treater helper (21).

5. System according to claim 1, wherein said digital controller (16) of said bioprocess machine (13) includes a file (20) containing a description of each said automation function which can be a said provided capability and said digital controller (22) of said machine helper (14) includes a file (26) containing a description of each said response-on-request function which can be a said consumed capability.

6. System according to claim 1, wherein the DNP manager (19) of said bioprocess machine (13) and the DNP manager (25) of said machine helper (14) are configured for cooperating over said network (12) for establishing a paired condition.

7. System according to claim 1, wherein said system devices include a plurality of said machine helpers (14) and the DNP manager (19) of said bioprocess machine (13) is configured for establishing a said paired condition simultaneously with at least one said machine helper(s) (14).

8. System according to claim 1, wherein said system devices include a first said machine helper (14) and a second said machine helper (14); the fluid treater helper (21) of the first machine helper (14) and the fluid treater helper (21) of the second machine helper (14) are configured for being physically coupled one another; and the DNP manager (25) of said first machine helper (14) and the DNP manager (25) of said second machine helper (14) are configured for cooperating over said network (12) for establishing a paired condition; wherein the recipe manager (51) of the first machine helper (14) and the recipe manager (51) of the second machine helper (14) are configured such that in said paired condition:
the recipe manager (51) of the first machine helper (14) has at least one provided capability that it does not have when not in paired condition, said provided capability being a response-on-request function controlling and/or testing an operating parameter of the treater helper (21) of the second machine helper (14) or testing a physico-chemical or biological quantity of said fluid sensed by the treater helper (21) of the second machine helper (14); and
the recipe manager (51) of the second machine helper (14) has at least one consumed capability that it does not have when not in paired condition, wherein: if said provided capability is said response-on-request function controlling and/or testing an operating parameter of the treater helper (21) of the second machine helper (14) said consumed capability is a response-on-request function controlling and/or making available said operating parameter, and if said provided capability is said response-on-request function testing and/or controlling a physico-chemical or biological quantity of said fluid sensed by the treater helper (21) of the second machine helper (14) said consumed capability is a response-on-request function or deliver-on-request function making available said physico-chemical or biological quantity.

9. System according to claim 8, wherein the recipe manager (50) of said bioprocess machine (13), the recipe manager (51) of said first machine helper (14) and the recipe manager (51) of said second machine helper (14) are configured such that in said paired condition of the bioprocess machine (13) with the first machine helper (14) while the first machine helper (14) is in paired condition with the second machine helper (14) and while the bioprocess machine (13) is in paired condition with the second machine helper (14), the recipe manager (50) of the bioprocess machine (13) has the same provided capability as the provided capability in the first machine helper (14), said same provided capability and said provided capability in the first machine helper (14) corresponding to said consumed capability in the second machine helper (14).

10. System according to claim 8, wherein the recipe manager (50) of said bioprocess machine (13) and the recipe manager (51) of said first machine helper (14) are configured such that in said paired condition of the bioprocess machine with the first machine helper (14) while the first machine helper (14) is in paired condition with the second machine helper (14), the reporting manager (60) of the bioprocess machine (13) has a provided capability replicating the provided capability in the first machine helper (14) corresponding to the consumed capability in the second machine helper (14).

11. System according to claim 8, wherein the recipe manager (50) of said bioprocess machine (13) and the recipe manager (51) of said first machine helper (14) are configured such that in said paired condition of the bioprocess machine with the first machine helper (14) while the first machine helper (14) is in paired condition with the second machine helper (14), the recipe manager (50) of the bioprocess machine (13) has a provided capability embedding the provided capability in the first machine helper (14) corresponding to the consumed capability in the second machine helper (14).

12. System according to claim 1, wherein said system devices include a first said bioprocess machine (13), a second said bioprocess machine (13) and a plurality of said machine helpers (14); and the DNP manager (25) of at least one said machine helper (14) is configured for establishing a said paired condition either with said first bioprocess machine (13) or with said second bioprocess machine (13).

13. System according to claim 1, having a first said machine helper (14) in which the consumed capability is a response-on-request function controlling and/or making available an operating parameter of the treater helper (21) of said first machine helper (14); and having a second said machine helper (14) in which the consumed capability is a response-on-request function making available a physico-chemical or biological quantity of the fluid sensed by the treater helper (21) of second machine helper (14), wherein the first machine helper (14) is a pump in which the consumed capability is a request-on-response function controlling and/or making available a speed of the pump and the second machine helper (14) is a pH sensor or a flow sensor in which the consumed capability is a response-on-request function making available a pH of said fluid or a flow of said fluid.

14. System according to claim 1, wherein each said MtoM communication tool (18, 24) is configured for connecting to a said network (12) which is a network with Internet Protocol, such as Ethernet, Wi-Fi, Bluetooth or cellular 5G.

15. System according to claim 1, wherein:
said digital controller (16) of said bioprocess machine (13) and said digital controller (22) of said machine helper (14) each further include a graphical user interface manager (17, 23) (GUI manager);
the GUImanager (17) of the bioprocess machine (13) and the GUImanager (23) of the machine helper (14) are configured such that in said paired condition:
the GUI manager (17) of the bioprocess machine (13) has at least one provided capability that it does not have when not in paired condition, said provided capability being an interface function controlling and/or displaying an operating parameter of said treater helper (21) or displaying a physico-chemical or biological quantity of said fluid sensed by said treater helper (21); and
the GUI manager (23) of the machine helper (14) has at least one consumed capability which is modified with respect to when not in paired condition, wherein: if said provided capability is said interface function controlling and/or displaying an operating parameter of the treater helper (21) said consumed capability is an interface function controlling and/or displaying said operating parameter, and if said provided capability is said interface function displaying a physico-chemical or biological quantity of said fluid sensed by the treater helper (21) said consumed capability is an interface function displaying said physico-chemical or biological quantity.
